(19) United States
(12) Patent Application Publication
(10) Pub. No.: US 2018/0116810 A1
(43) Pub. Date: May 3, 2018

(54) TENSEGRITY JOINTS

(71) Applicant: Articulus Bio, LLC, Boulder, CO (US)

(72) Inventor: Jerome Rifkin, Louisville, CO (US)

(21) Appl. No.: 15/715,142

(22) Filed: Sep. 25, 2017

Related U.S. Application Data
(60) Provisional application No. 62/399,063, filed on Sep. 23, 2016.

(51) Int. Cl. A61F 2/38; A61F 2/30

(52) U.S. Cl. CPC A61F 2/3877; A61F 2/3859; A61F 2/461; A61F 2/30749; A61F 2002/3881; A61F 2/389

(57) ABSTRACT

An articulating joint includes a base member, a suspended member and at least one tension member. The base member includes at least two base member peripheral anchors and a base member intermediate anchor located between the base member peripheral anchors. The suspended member includes at least two suspended member peripheral anchors and a suspended member intermediate anchor located between the suspended member peripheral anchors. The at least one tension member couples the base member peripheral anchors to the suspended member peripheral anchors and couples the base member intermediate anchor to the suspended member intermediate anchor so that the base member and the suspended member are configured for constrained relative articulation with the suspended member partially received within the base member.

### CROSS REFERENCE TO RELATED APPLICATIONS

This application claims the priority benefit of U.S. Provisional Application No. 62/399,063 filed on Sep. 23, 2016 which is incorporated herein by reference in its entirety.

### SUMMARY

The disclosure further seeks to provide a knee joint having a patellar component including first, second, third and fourth patellar component peripheral anchors and first and second patellar component intermediate anchor located between the patellar component peripheral anchors; a femoral component including at least one lumen therethrough and first and second femoral component intermediate anchors; a tibial component including at least one lumen therethrough and first and second tibial component intermediate anchors; and at least one tension member coupling the first and second patellar component peripheral anchors to one another through the lumen of the femoral component, coupling the third and fourth patellar component peripheral anchors to one another through the lumen of the tibial component, coupling the first femoral component intermediate anchor to the first tibial component member intermediate anchor, coupling the second femoral component intermediate anchor with the second tibial component intermediate anchor so that the femoral component and the tibial component are configured for constrained articulation relative to the patellar component and are partially received thereby.

The disclosure seeks to provide an articulating joint with a base member including at least two base member peripheral anchors and a base member intermediate anchor located between the base member peripheral anchors, a suspended member including at least two suspended member peripheral anchors and a suspended member intermediate anchor located between the suspended member peripheral anchors and at least one tension member coupling the base member peripheral anchors to the suspended member peripheral anchors and coupling the base member intermediate anchor to the suspended member intermediate anchor so that the base member and the suspended member are configured for constrained relative articulation with the suspended member partially received within the base member.

The disclosure additionally seeks to provide an articulating joint having a first suspended member including at least one lumen therethrough and a suspended member intermediate anchor, a base member configured to receive a portion of the first suspended member and including at least two base member peripheral anchors and at least one base member intermediate anchor located between the base member peripheral anchors and at least one tension member threaded through the base member and suspended member intermediate anchors and coupling the base member peripheral anchors to one another through the lumen of the suspended articulating member so that the base member and the suspended member are configured for constrained relative articulation with the suspended member partially received within the base member.

### BRIEF DESCRIPTION OF THE FIGURES

The summary above, as well as the following detailed description of illustrative embodiments, is better understood when read in conjunction with the appended drawings. For the purpose of illustrating the present disclosure, example constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIGS. 1A-1E illustrate an example femoral component usable as part of an example spherical joint in accordance with embodiments of the disclosure.

FIGS. 1F-1J illustrate an example acetabular component usable as part of an example spherical joint in accordance with embodiments of the disclosure.

FIG. 1K illustrates an example spherical joint in accordance with embodiments of the disclosure.

FIGS. 2A & 2B illustrate example tension members usable as part of an example spherical joint in accordance with embodiments of the disclosure.

FIGS. 3A-3F illustrate an example patellar component usable as part of an example knee joint in accordance with embodiments of the present disclosure.

FIGS. 3G-3I illustrate an example knee joint in accordance with embodiments of the present disclosure.

FIGS. 4A-4C illustrate a number of example tension members usable as part of an example knee joint in with embodiments of the present disclosure.

FIGS. 4D-4H illustrate an example femoral/tibial shaft component usable as part of an example knee joint in with embodiments of the present disclosure.

FIGS. 5A-5F illustrate an example shaft component usable as part of an example saddle joint in accordance with embodiments of the present disclosure.

FIGS. 5G-5M illustrate an example socket component usable as part of an example saddle joint in accordance with embodiments of the present disclosure.

FIGS. 5N-5R illustrate an example saddle joint in accordance with embodiments of the present disclosure.

FIGS. 6A-6J illustrate a number of example tension members usable as part of an example saddle joint in accordance with embodiments of the present disclosure.

FIGS. 7A-7D illustrate an example tensegrity hip joint in accordance with embodiments of the present disclosure.

FIG. 8 illustrates a first example simplified joint geometry.

FIG. 9 illustrates a second example simplified joint geometry.

FIG. 10 illustrates a third example simplified joint geometry.

FIG. 11 illustrates a fourth example simplified joint geometry.

### DETAILED DESCRIPTION

The following detailed description illustrates embodiments of the present disclosure and manners by which they can be implemented. Although the best mode of carrying out the present disclosure has been disclosed, those skilled in the art would recognize that other embodiments for carrying out or practicing the present disclosure are also possible.

It should be noted that the terms “first”, “second”, and the like, herein do not denote any order, quantity, or importance, but rather are used to distinguish one element from another. Further, the terms “a” and “an” herein do not denote a limitation of quantity, but rather denote the presence of at least one of the referenced item.

In machinery, currently joints use bearings to support weight and protect the relatively fragile or ponderously heavy pin and spherical joint mechanisms. This highly constrained joint design forces the adjacent limb segments to rotate around the joint, rather than around the center of mass of the limb segment. This consumes energy that could be conserved if the movement was more compliant.

In various embodiments, there are provided a series of joints that replace compressive/sliding interfaces in load bearing joints with tensile/wrapping structures. The result is a structure stronger per unit weight, and also more compliant overall movement.

Embodiments of the present disclosure substantially eliminate, or at least partially address, problems in the prior art, enabling reduction in sliding wear, reduction in debris, increased longevity, and lighter weight for joint devices. These devices may include, but are not limited to, spherical joints, hip joints, knee joints, saddle joints and ankle joints. When implemented in robotic devices, features may include, but are not limited to, light weight, improved load bearing capacity, and compliant movement. Disclosed embodiments may be either or both safer for human interaction and more stable on uneven terrain.

Additional aspects, advantages, features and objects of the present disclosure will be made apparent from the drawings and the detailed description of the illustrative embodiments construed in conjunction with the appended claims that follow.

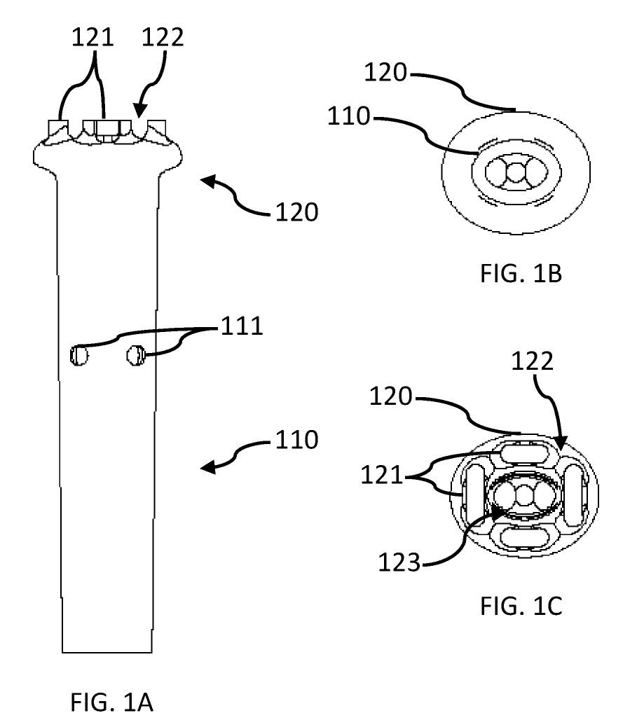

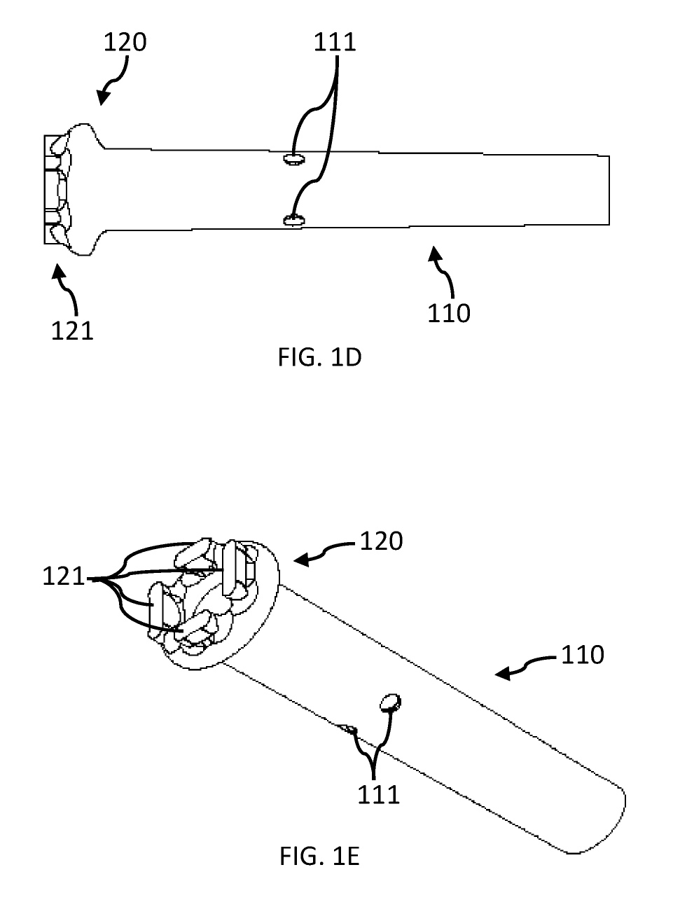

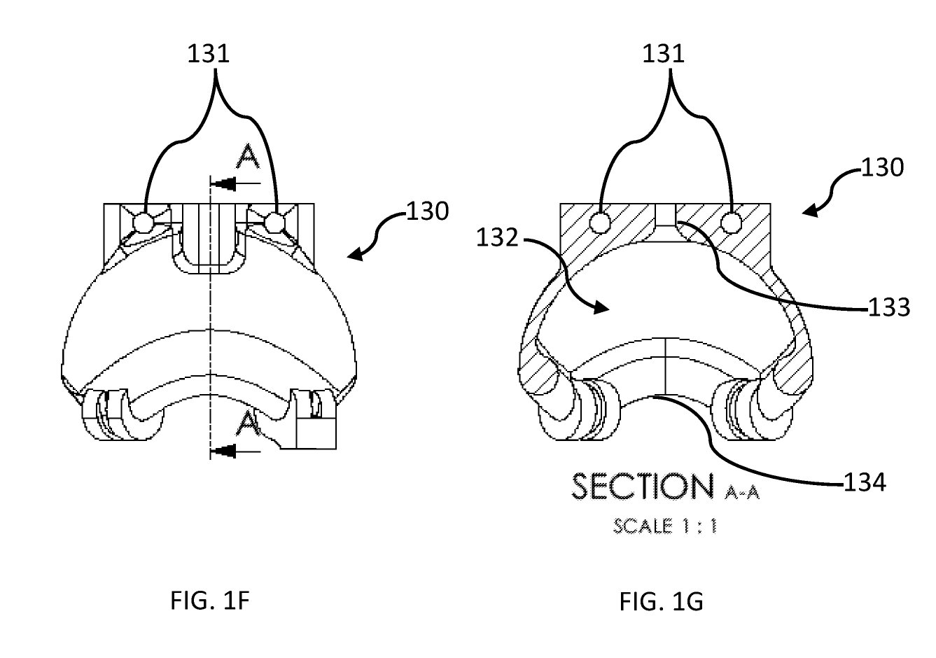

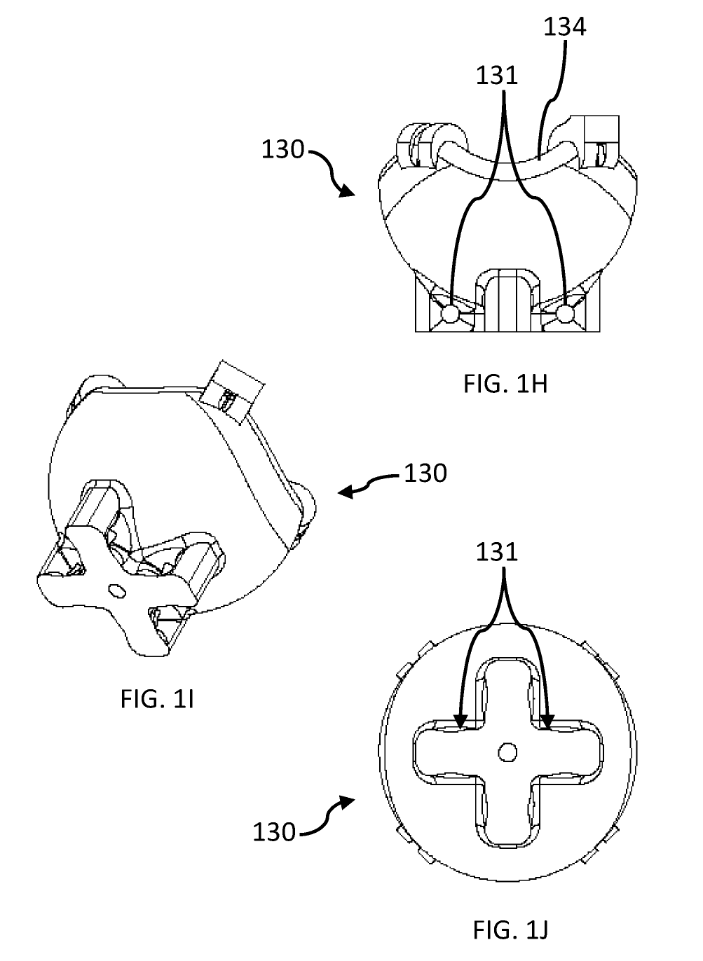

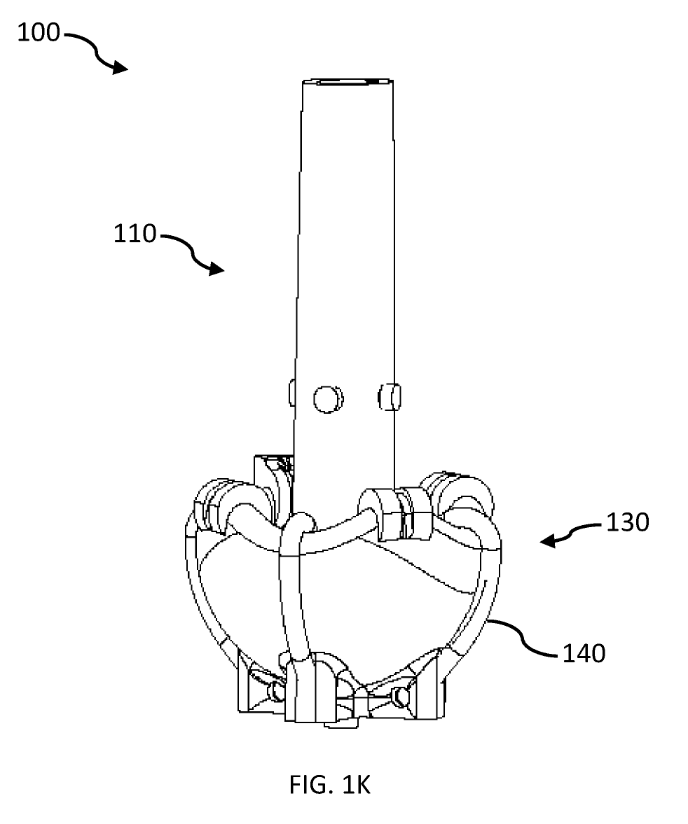

FIGS. 1 & 2 illustrate an example spherical joint in accordance with embodiments of the disclosure which may be configured to function as a hip or shoulder joint. Spherical joint 100 includes a femoral component and an acetabular component 130. The femoral component includes a femoral shaft 110 and a femoral head 120 and having first, second, third and fourth femoral component peripheral anchors 111 and a femoral component intermediate anchor 123 located substantially central to the femoral component peripheral anchors 111. Femoral shaft 110 may comprise a hollow tube with any of a variety of cross sectional shapes including but not limited to ovular.

Acetabular component 130 is configured to receive at least a portion of femoral head 120 within an interior 132 and includes first, second, third and fourth acetabular component peripheral anchors 131 and an acetabular component intermediate anchor 133 located substantially central to acetabular component peripheral anchors 131.

At least one tension member 150 couples the first acetabular component peripheral anchor to the first femoral component peripheral anchor, the second acetabular component peripheral anchor to the second femoral component peripheral anchor, the third acetabular component peripheral anchor to the third femoral component peripheral anchor, the fourth acetabular component peripheral anchor to the fourth femoral component peripheral anchor. In an example, acetabular component intermediate anchor 131 is coupled with femoral component intermediate anchor 123 such that femoral head 120 is prevented from contacting acetabular component 130.

Femoral head 120, which has rounded edges over which tension member 150 will bend while bearing load, includes a plurality of projections 121 protruding therefrom and defining therebetween notches configured to receive tension member 150. Protrusions 121 may take any of a variety of forms including but not limited to cylindrical or roughly cylindrical. In an example, two projections 121 are provided for each tension member or tension member segment anchored to acetabular component 130 and each projection 121 exhibits a maximal radius where they will contact one or more tension members and guide their wrapping.

Engagement of tension member 150 with protrusions 121 constrains twisting of femoral head 120 relative to acetabular component 130 about a central longitudinal axis of the femoral component. Within the restricted range of motion, twisting of femoral head 120 relative to acetabular component 130 about the central longitudinal axis distracts the femoral component from acetabular component 130 which is resisted and balanced by tension member 150 constraining movement between femoral component intermediate anchor 123 and acetabular component intermediate anchor 133.

While any of a variety of configurations may be implemented, in an example, the first and third acetabular component peripheral anchors 131 are substantially diametrically opposed and the second and fourth acetabular component peripheral anchors 131 are substantially diametrically opposed. In a further example, adjacent acetabular component peripheral anchors are separated from one another by an arc measuring between about 80 and about 100 degrees of the periphery of the acetabular component 130. For example, the first and second acetabular component peripheral anchors 131 are separated from one another by an arc measuring about 90 degrees of a periphery of acetabular component 130, the second and third acetabular component peripheral anchors are separated from one another by an arc measuring about 90 degrees of a periphery of acetabular component 130, the third and fourth acetabular component peripheral anchors are separated from one another by an arc measuring about 90 degrees of a periphery of acetabular component 130 and the fourth and first acetabular component peripheral anchors are separated from one another by an arc measuring about 90 degrees of a periphery of the acetabular component 130.

In an example, the first and third femoral component peripheral anchors 131 are substantially diametrically opposed and the second and fourth femoral component peripheral anchors 131 are substantially diametrically opposed. In a further example, adjacent femoral component peripheral anchors are separated from one another by an arc measuring between about 80 and about 100 degrees. For example, the first and second femoral component peripheral anchors 111 are separated from one another by an arc measuring about 90 degrees of a periphery of the femoral component, the second and third femoral component peripheral anchors 111 are separated from one another by an arc measuring about 90 degrees of a periphery of the femoral component, the third and fourth femoral component peripheral anchors 111 are separated from one another by an arc measuring about 90 degrees of a periphery of the femoral component and the fourth and first femoral component peripheral anchors 111 are separated from one another by an arc measuring about 90 degrees of a periphery of the femoral component.

The edges of acetabular component 130 include four semicircular depressions 134 in which tension members, naturally seeking a local energy minimum, are received and cannot skip from one to the other. Further, the edge of socket component 130 features a rounded edge over which tension members will bend as they bear load.

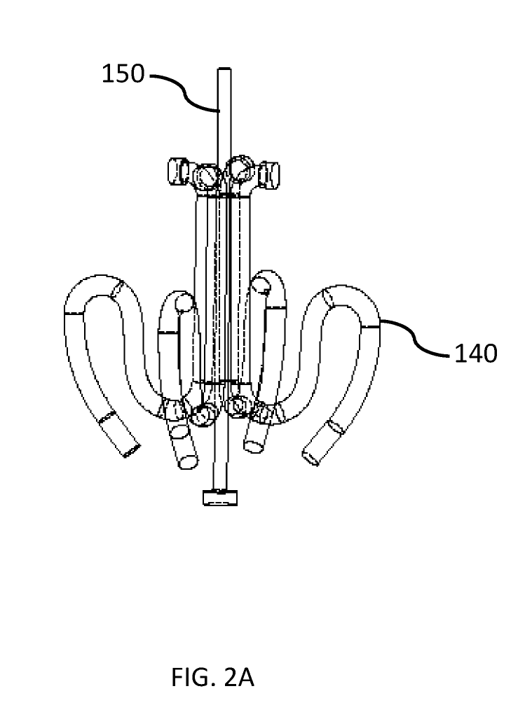

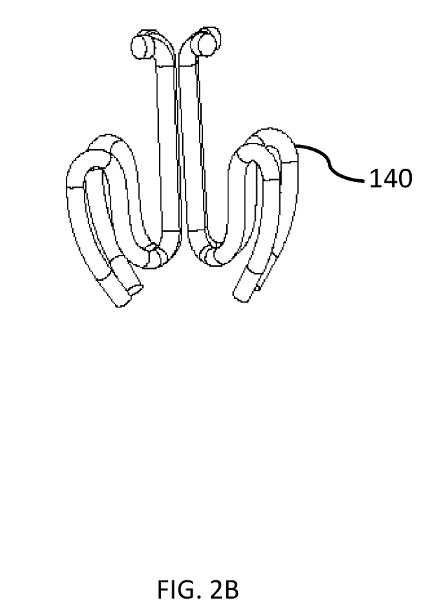

Referring to FIG. 2A, tension member 150 which is threaded between femoral head intermediate anchor 123 to which it is secured at one end and to acetabular component intermediate anchor 133 at the other end and is configured to constrain distraction of femoral head 120 from acetabular component 130. The cooperation of tension member 140 with notches 121 and the associated projections constrains twisting of the femoral component with respect to acetabular component 130 and constrains tilting or rotation of the femoral component with respect to the acetabular component 130 about a transverse axis.

Tension member 150 of spherical joint 100, may take any of a variety of configurations and may include a first tension member coupling the first acetabular component peripheral anchor to the first femoral component peripheral anchor, a second tension member coupling the second acetabular component peripheral anchor to the second femoral component peripheral anchor, and a third tension member coupling the third acetabular component peripheral anchor to the third femoral component peripheral anchor and a fourth tensioning member coupling the fourth acetabular component peripheral anchor to the fourth femoral component peripheral anchor. Each of these tension members follows a similar path: connecting one end to a socket component peripheral anchor 131, wrapping up the external surface of socket component 130, over the lowest point of the nearest energy well 134, down into interior 132 of socket component 130, wrapping a lip on femoral head 120, between two flanking projections 121, up inside shaft 110, out of shaft 110 through nearest femoral component peripheral anchor 111, and terminating at that anchor in a knot or other connection mechanism common in the field of tension member termination.

Further, a fifth tension member may be provided configured for coupling the femoral component intermediate anchor 123 with the acetabular component intermediate anchor 133. The fifth tension member passes from socket component 130 to shaft 110, but without any wrapping. From the center of socket component 130, the fifth tension member traverses directly to shaft 110, travels up the center thereof, and terminates on femoral component intermediate anchor 123 such that shaft 110 is restricted in movement away from socket component 130. The fifth tension member is also configured to keep the first through fourth tension members pulled taut, so they cannot dislocate from femoral head 120. By adjusting the tension in the fifth tension member, the stiffness of the joint motion can be modulated.

Tension members disclosed herein throughout may be formed from sheathed ropes for improved abrasion resistance. Suitable sheathed ropes include but are not limited to Dyneema™ and Spectra™ brand rope fibers.

When the femoral component moves relative to acetabular component 130 in a tipping motion, as also happens to the femoral head relative to the socket in abduction and adduction, one or more of wrapping tension members further wraps the lip of femoral head 120, pulling femoral head 120 away from acetabular component 130, putting the fifth tension member under load, or increased load, depending on the initial tension of the joint. When the fifth tension member reaches equilibrium with the force of the motion, movement stops. Using very high elastic modulus tension members to couple femoral component intermediate anchor 123 with acetabular component intermediate anchor 133 leads to a well-controlled range of motion in tipping.

In an example, when the peripheral anchors of the femoral and acetabular components are separated by arcs measuring 90 degrees, tension members anchored thereto may be offset from preferred axes of rotation for tipping by 45 degrees such dynamic instability is avoided in the case when tipping occurs directly toward a tension member. Otherwise, a local energetic maxima occurs with are lower energy states on either side of the tension member and there will be a natural tendency for a lower energy configuration to be occupied.

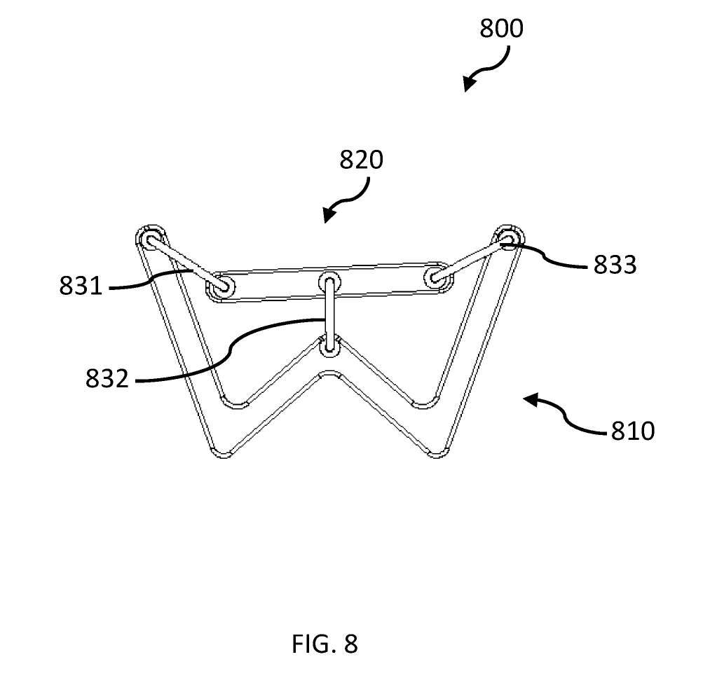

An articulating joint in the form of a spherical joint may be constructed from one or more simplified geometries. Referring to FIG. 8, a first example of one such simplified geometry includes a base member 810, a suspended member 820 and at least one tension member. In an further example, the at least one tension member comprises first 831, second 832 and third 833 tension members. Base member 810 includes at least two base member peripheral anchors and a base member intermediate anchor located between the base member peripheral anchors. Suspended member 820 includes at least two suspended member peripheral anchors and a suspended member intermediate anchor located between the suspended member peripheral anchors. Tension member 831 couples a first of the base member peripheral anchors to a first of the suspended member peripheral anchors, tension member 833 couples a second of the base member peripheral anchors to a second of the suspended member peripheral anchors and tension member 832 couples the base member intermediate anchor to the suspended member intermediate anchor so that base member 810 and suspended member 820 are configured for constrained relative articulation with suspended member 820 partially received within base member 810.

In another example, the first simplified geometry may be multiplied and combined with itself such that the at least two base member peripheral anchors include first, second, third and fourth base member peripheral anchors and the at least two suspended member peripheral anchors include first, second, third and fourth suspended member peripheral anchors. In an orthogonal combination, the first and third base member peripheral anchors may be substantially diametrically opposed, the second and fourth base member peripheral anchors may be substantially diametrically opposed, the first and third suspended member peripheral anchors may be substantially diametrically opposed and the second and fourth suspended member peripheral anchors may be substantially diametrically opposed. Each adjacent pair of base member peripheral anchors are separated from one another by an arc measuring substantially 90 degrees. For example, the first base member peripheral anchor may be spaced from the fourth base member peripheral anchor by an arc measuring in the range from about 80 degrees to about 100 degrees. Each adjacent pair of suspended member peripheral anchors are also separated from one another by an arc measuring about 90 degrees. For example, the first suspended member peripheral anchor may be spaced from the fourth suspended member peripheral anchor by an arc measuring in the range from about 80 degrees to about 100 degrees.

In this first example combination, at least one tension member couples the first base member peripheral anchor to the first suspended member peripheral anchor, couples the second base member peripheral anchor to the second suspended member peripheral anchor, couples the third base member peripheral anchor to the third suspended member peripheral anchor, and couples the fourth base member peripheral anchor to the fourth suspended member peripheral anchor.

In another example multiplication and combination of the first simplified geometry the at least two base member peripheral anchors include first, second, third and fourth base member peripheral anchors; the at least two suspended member peripheral anchors include first, second, third and fourth suspended member peripheral anchors; and the at least one suspended member intermediate anchor includes first, second, third and fourth suspended member intermediate anchors. The at least one tension member couples the first base member peripheral anchor to the first suspended member peripheral anchor, couples the second base member peripheral anchor to the second suspended member peripheral anchor, couples the third base member peripheral anchor to the third suspended member peripheral anchor, couples the fourth base member peripheral anchor to the fourth suspended member peripheral anchor, and couples the first, second third and fourth suspended member intermediate anchors to the at least one base member intermediate anchor.

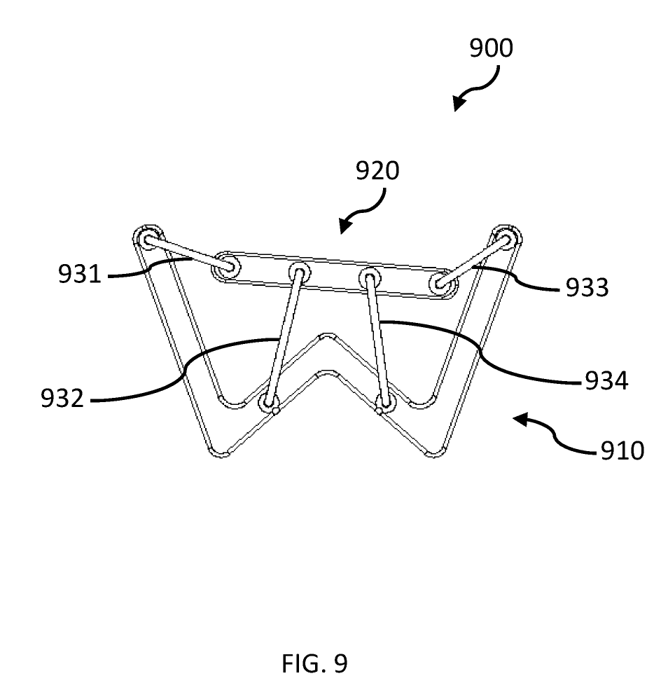

Referring to FIG. 9, a second example of a simplified geometry, includes a base member 910, a suspended member 20 and at least one tension member. In an further example, the at least one tension member comprises first 931, second 932, third 933 and fourth 934 tension members. Base member 910 includes at least two base member peripheral anchors and two base member intermediate anchors located between the base member peripheral anchors. Suspended member 920 includes at least two suspended member peripheral anchors and two suspended member intermediate anchors located between the suspended member peripheral anchors. First tension member 931 couples a first of the base member peripheral anchors to a first of the suspended member peripheral anchors, second tension member 933 couples a second of the base member peripheral anchors to a second of the suspended member peripheral anchors, third tension member 932 couples the first base member intermediate anchor to the first suspended member intermediate anchor and fourth tension member 934 couples the second base member intermediate anchor with the second suspended member intermediate anchor so that base member 910 and suspended member 920 are configured for constrained relative articulation with suspended member 920 partially received within base member 910.

As with all disclosed embodiments, tension members loops made from flexible cordage that is splice, knotted, woven, sewn, swaged or a combination of these as known in the art of working with rope, filament, cable, flat braid and other similar constructions.

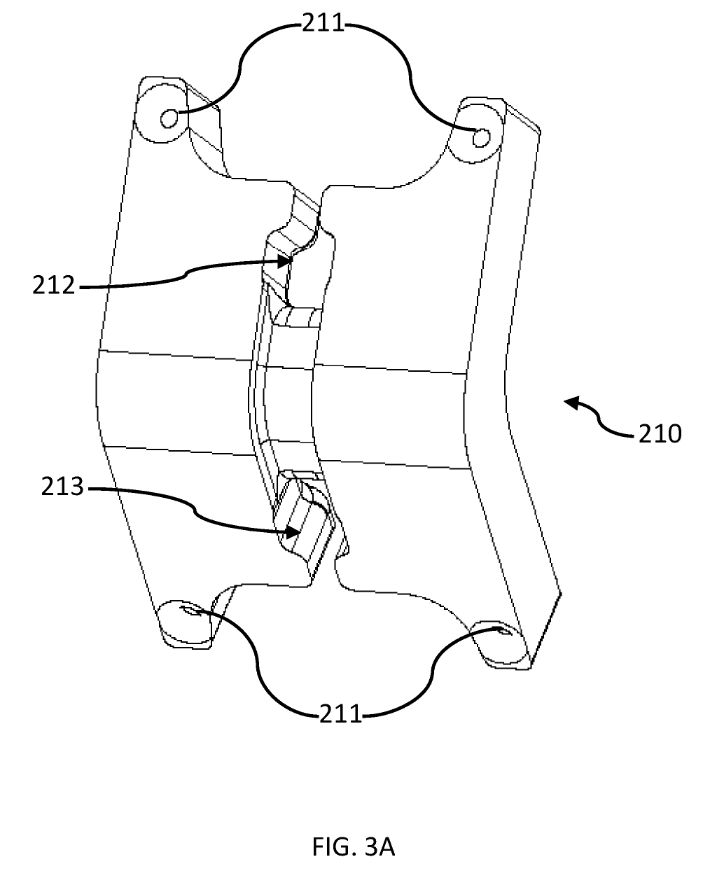

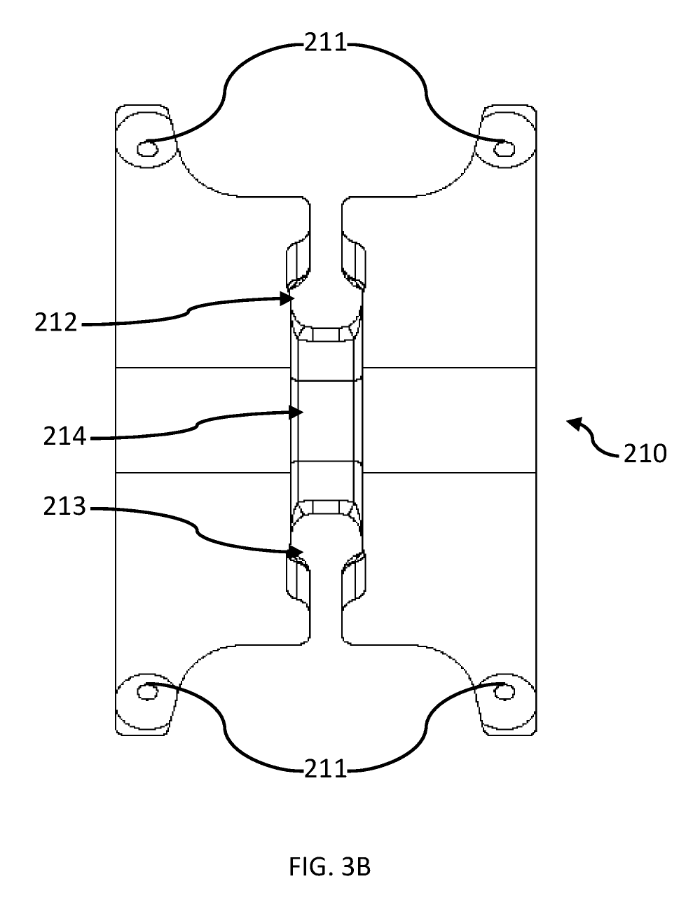

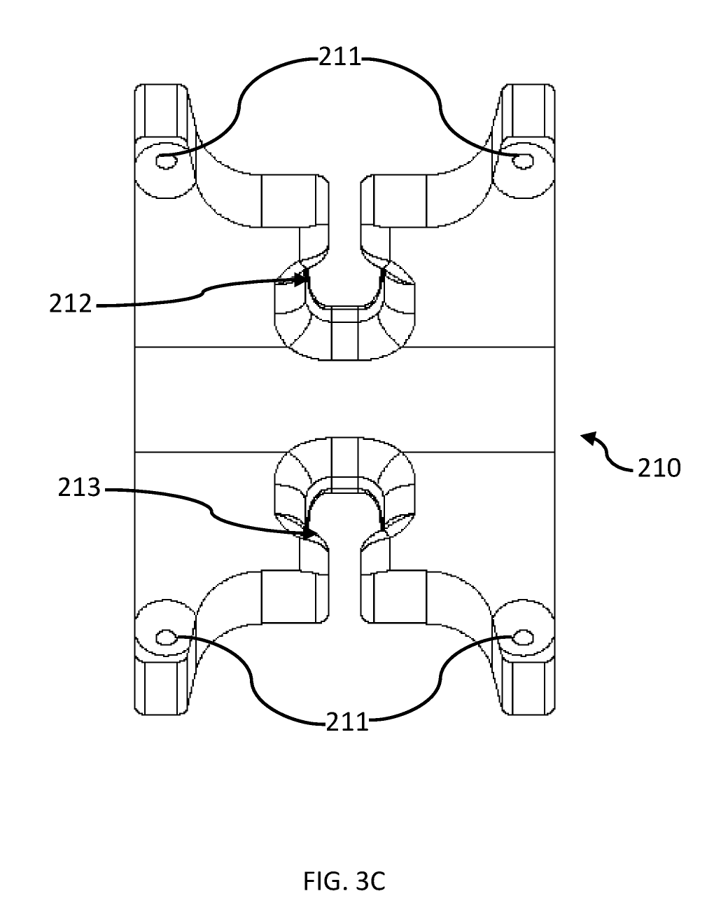

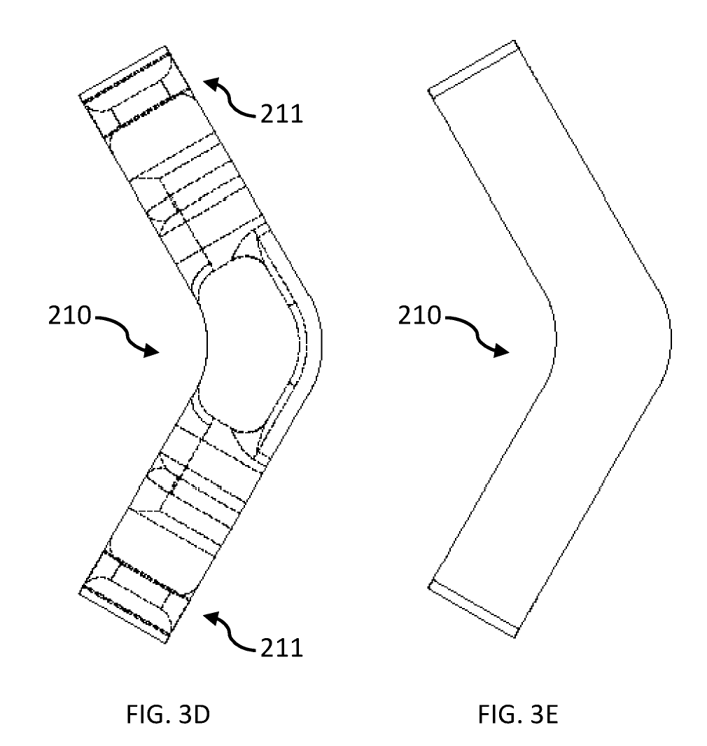

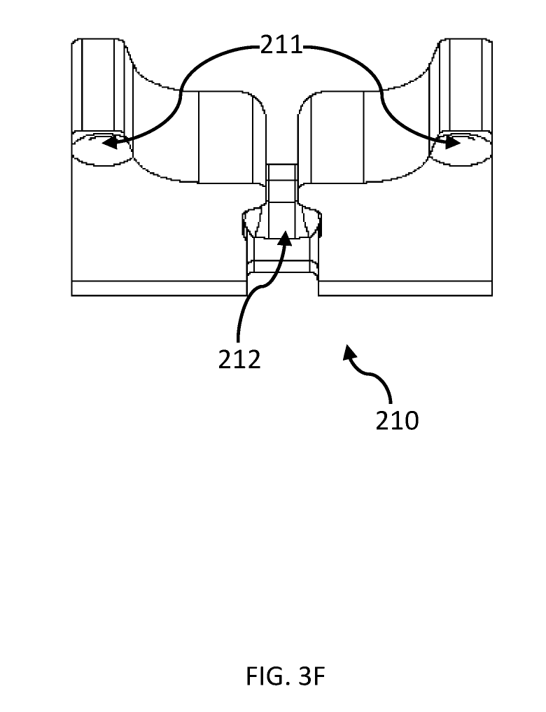

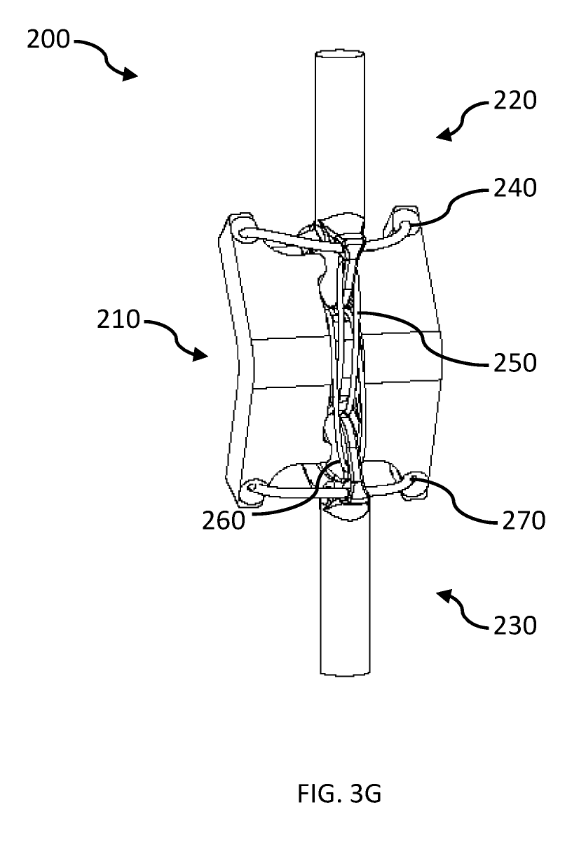

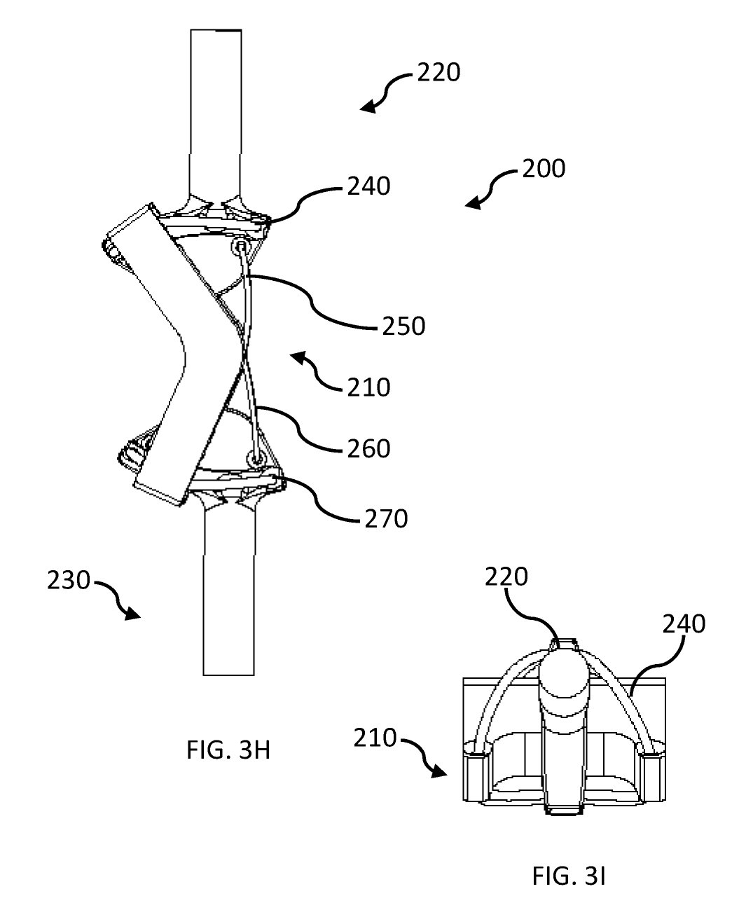

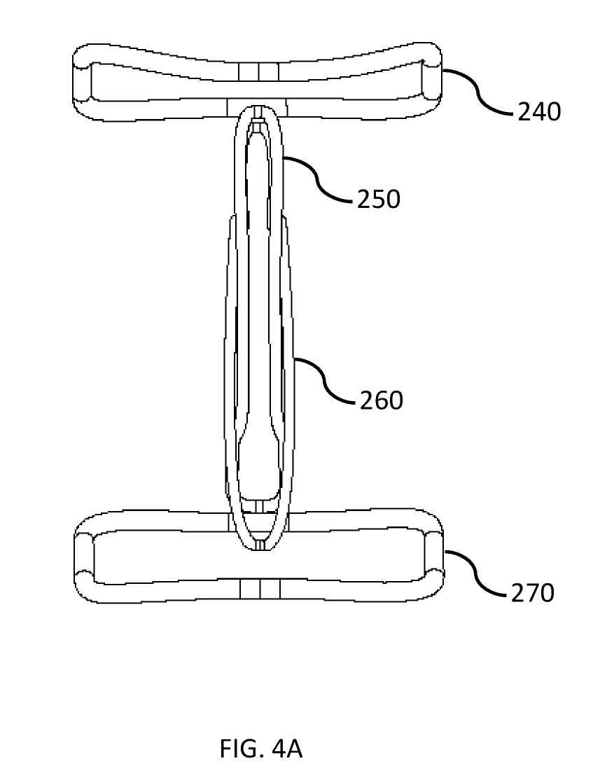

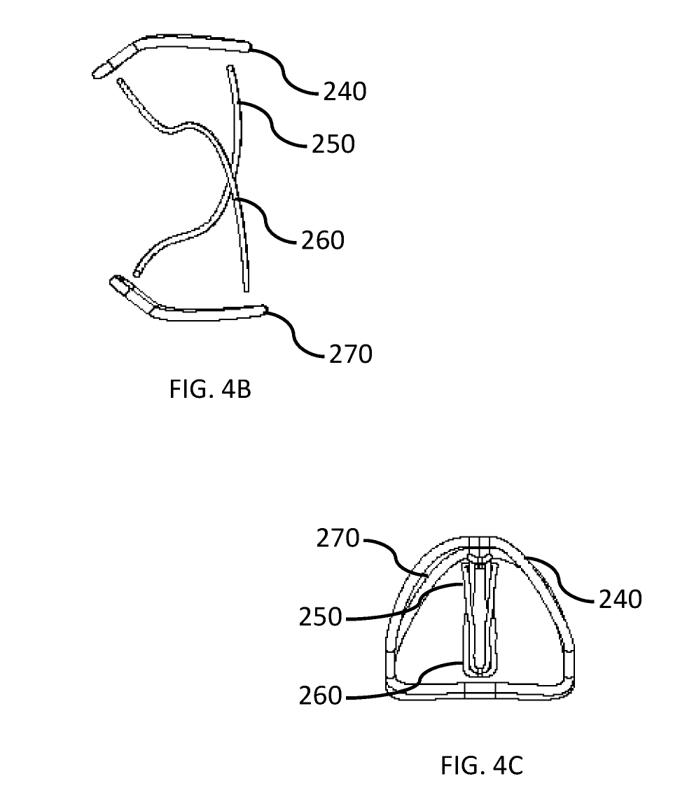

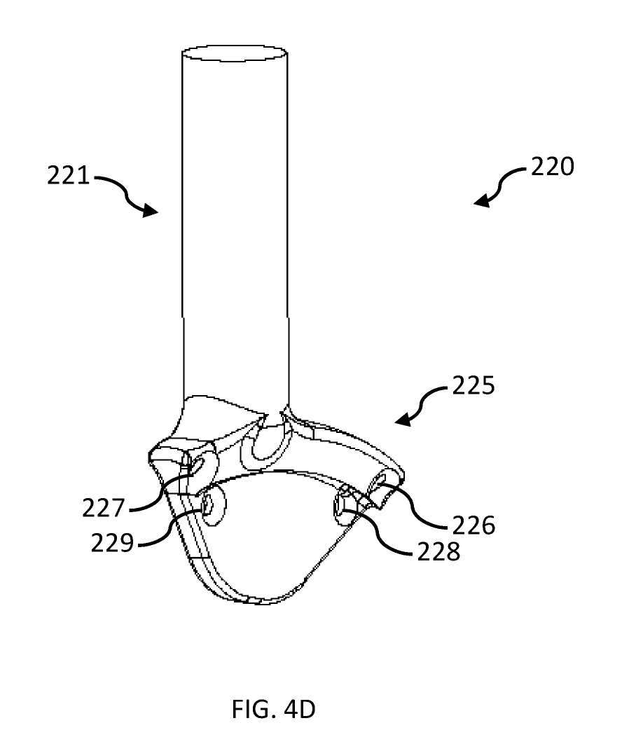

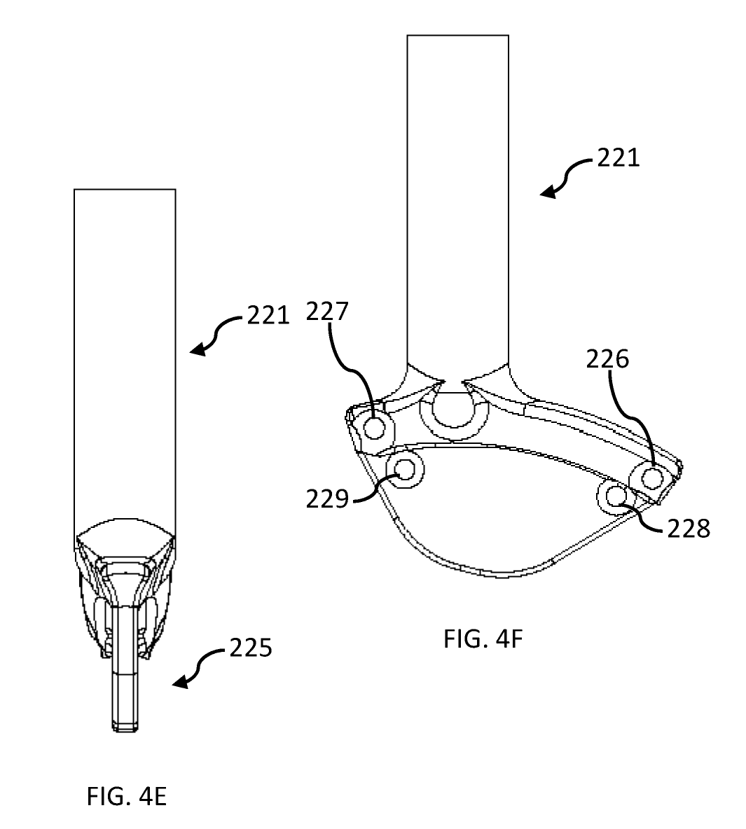

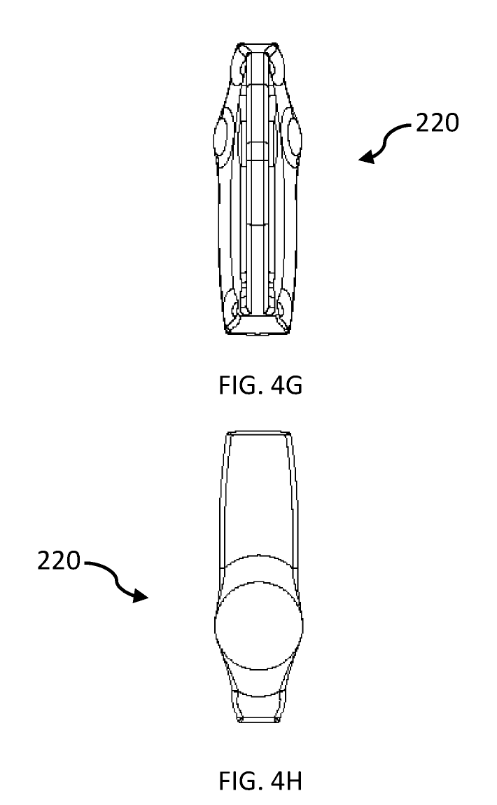

FIGS. 3 & 4 illustrate an example knee joint 200 in accordance with embodiments of the present disclosure having a polycentric, complaint joint structure. Knee joint 200 includes a patellar component or kneecap articulation component 210, a femoral or suspended component 220 and a tibial suspended component 230. Patellar component 210 exhibits symmetry between left and right side and between upper and lower ends and includes first, second, third and fourth patellar component peripheral anchors 211. The upper and lower portions of patellar component 210 each include a slot 212 and 213 on a central plane. A groove 214 in patellar component 210 is configured to receive tension members 25 and 260 (described below).

Femoral component or shaft 220 includes a blade portion 225 having first and second lumens 226 and 227 therethrough and first and second femoral component intermediate anchors 228 and 229. Tibial component or shaft 230, which includes all of the features of the femoral component. As such, tibial component 230 includes at least one lumen analogous to lumen 226 therethrough and first and second tibial component intermediate anchors analogous to anchors 228 and 229. In the assembly, the interaction of blade portion 225 of the femoral and tibial components 220 and 230 and upper and lower and lower slots 212 and 213 of patellar component 210, respectively, limit rotation around a substantially vertical axis in use.

At least one tension member 240 coupling the first and second patellar component peripheral anchors 211 to one another through lumen 226 of femoral component 220, coupling the third and fourth patellar component peripheral anchors 211 to one another through lumen 226 of tibial component 230, coupling the first femoral component intermediate anchor 228 to the second tibial component member intermediate anchor 229, coupling the second femoral component intermediate anchor 229 with the first tibial component intermediate anchor 228 so that femoral component 220 and tibial component 230 are configured for constrained articulation relative to patellar component 210 and are partially received thereby. Each anchor may be configured with rounded edges to reduce stress concentration and wear in tension members.

In an example, the at least one tension member may comprise a plurality of tension members. A first tension member 240 winds through first peripheral anchor 226 of femoral component 220, through a first peripheral anchor 211 of patellar component 210, through a second peripheral anchor 227 of femoral component 220 and then through a second peripheral anchor 211 of patellar component 210 and forms a continuous loop. First tension member 240 absorbs vertical load placed on femoral component 220, and transfers the load to patellar component 210.

A second tension member 250, which may be thought of as a cruciate tension member, winds through a first intermediate anchor 228 of femoral component 220, through a second intermediate anchor 213 of patellar component 210, through a second intermediate anchor 229 of tibial component 230, through the second intermediate anchor 213 of patellar component 210 and then through the first intermediate anchor 228 of femoral component 220 and forms a continuous loop. Second tension member 250 restricts distraction of the femoral and tibial components and also blocks anterioposterior joint buckling.

A third tension member 260, which may be thought of as another cruciate tension member, winds through a first intermediate anchor 228 of tibial component 230, through a first intermediate anchor 212 of patellar component 210, through a second intermediate anchor 229 of femoral component 220, through the second intermediate anchor 213 of patellar component 210 and then through the first intermediate anchor 228 of tibial component 220 and forms a continuous loop. Third tension member 260 also restricts distraction of the femoral and tibial components and also blocks anterioposterior joint buckling.

A fourth tension member 270 winds through first peripheral anchor 226 of a tibial component 230, through a third peripheral anchor 211 of patellar component 210, through a second peripheral anchor 227 of tibial component 230 and then through a second peripheral anchor 211 of patellar component 210 and also forms a continuous loop. As with first tension member 240, fourth tension member 270 absorbs vertical load placed on tibial component 230, and transfers the load to patellar component 210.

During movement of either femoral component 220 or tibial component 230 into either flexion or extension, tension members 250 and 260 cause the other of the femoral and tibial components to move in the same mode. Tension members 240, 250 and 260 resist distraction of femoral component 220 while tension members 250, 260 and 270 resist distraction of tibial component 230. Tension member 240 constrains both flexion and extension of femoral component 220 relative to patellar component 210 while tension member 270 constrains both flexion and extension of tibial component 230 relative to patellar component 210. In an example, patellar component 210 is configured with about a 60-degree angle between its upper and lower portions so that in a neutral configuration of the joint, the upper and lower tension members to be at an angle to each other in a “neutral” configuration.

Knee joint 200 is a compliant analog of the human knee joint which has a preferred axis of rotation, two additional much more limited axes of rotation and a very limited relative translation of the femoral and tibial components.

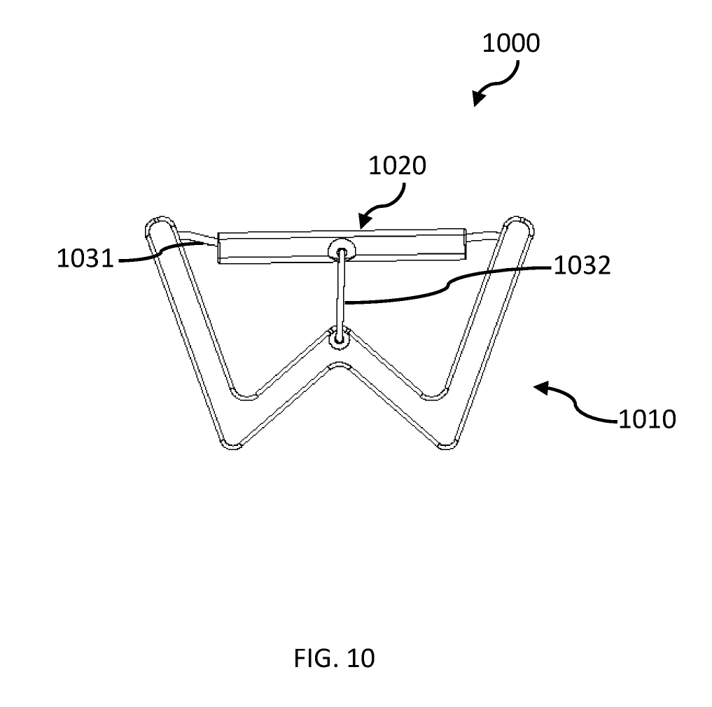

As with the spherical joint described above, an articulating joint in the form of a knee joint may be constructed/arranged/configured/conceptualized from one or more simplified geometries. Referring to FIG. 10, a second example of a simplified geometry includes a first suspended member 1020, a base member 1010 and at least one tension member. In an example the at least one tension member comprises a first tension member 1031 and a second tension member 1032. First suspended member 1020 includes at least one lumen therethrough and a suspended member intermediate anchor. Base member 1010 is configured to receive a portion of first suspended member 1020 and includes at least two base member peripheral anchors and at least one base member intermediate anchor located between the base member peripheral anchors. First tension member 1031 couples the base member peripheral anchors to one another through the lumen of suspended member 1020 so that base member 1010 and suspended member 1020 are configured for constrained relative articulation with suspended member 1010 partially received within base member 1020.

In another example, the second simplified geometry may be multiplied and combined with itself such that the at least two base member peripheral anchors include first, second, third and fourth base member peripheral anchors; the at least one lumen includes first and second lumens; and the at least one tension member couples the first base member peripheral anchor to the third base member peripheral anchor through the first lumen and couples the second base member peripheral anchor to the fourth base member peripheral anchor through the second lumen.

In another example multiplication and combination of the second simplified geometry, the at least two base member peripheral anchors include first, second, third, fourth, fifth, sixth, seventh and eighth peripheral anchors; the at least one lumen includes first, second, third and fourth lumens; and the at least one suspended member intermediate anchor includes first, second, third and fourth suspended member intermediate anchors. The at least one tension member couples the first and fourth base member peripheral anchors to one another through the first lumen, couples the second and seventh base member peripheral anchors to one another through the second lumen, couples the third and sixth base member peripheral anchors to one another through the third lumen, couples the fifth and eighth base member peripheral anchors to one another through the fourth lumen and couples the first, second, third and fourth suspended member intermediate anchors to the at least one base member intermediate anchor.

In another example combination, the second simplified geometry may be combined with at least one other disclosed geometry. The at least two base member peripheral anchors include first, second, third and fourth base member peripheral anchors and the suspended member includes a lumen therethrough. The at least one tension member couples the first and third base member peripheral anchors with the at least two suspended member peripheral anchors and couples the second and fourth base member peripheral anchors to one another through the suspended member lumen.

In still another example, the second simplified geometry may be multiplied and combined with itself such that the at least two base member peripheral anchors include first, second, third and fourth peripheral anchors; the at least one base member intermediate anchor includes first, second and third base member intermediate anchors; the at least one lumen includes first and second lumens; the at least one suspended member intermediate anchor includes first, second and third suspended member intermediate anchors; and the at least one tension member couples first and third base member peripheral anchors to one another through the first lumen, couples the second and fourth base member peripheral anchors to one another through the second lumen, couples the first base member intermediate anchor with the first suspended member intermediate anchor, couples the second base member intermediate anchor with the second suspended member intermediate anchor and couples the third base member intermediate anchor with the third suspended member intermediate anchor.

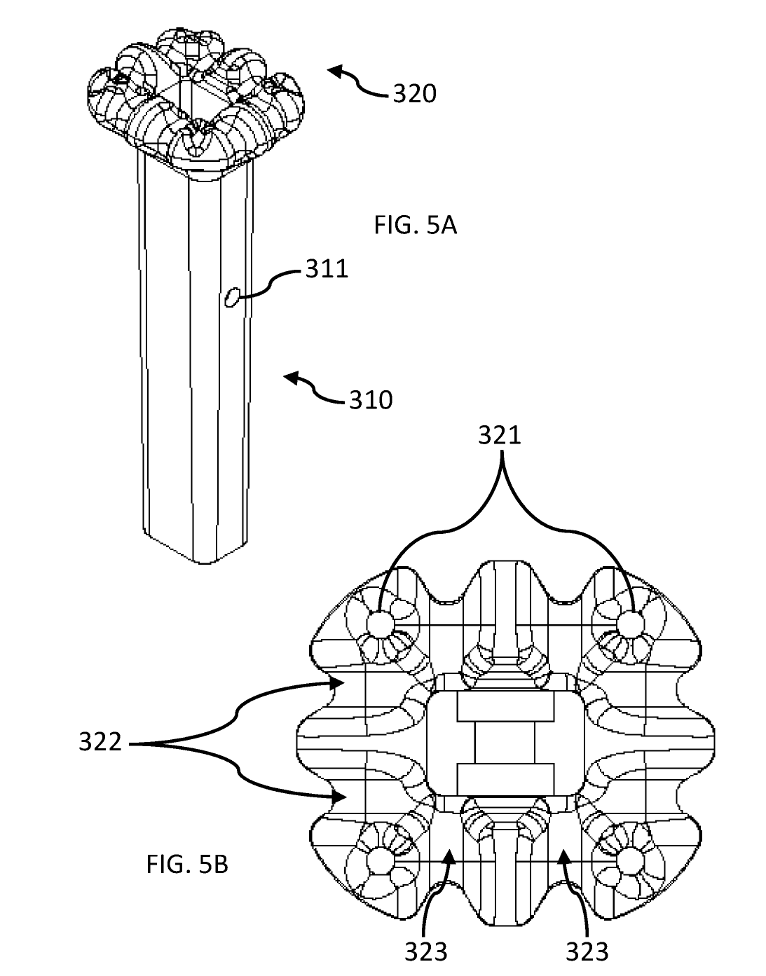

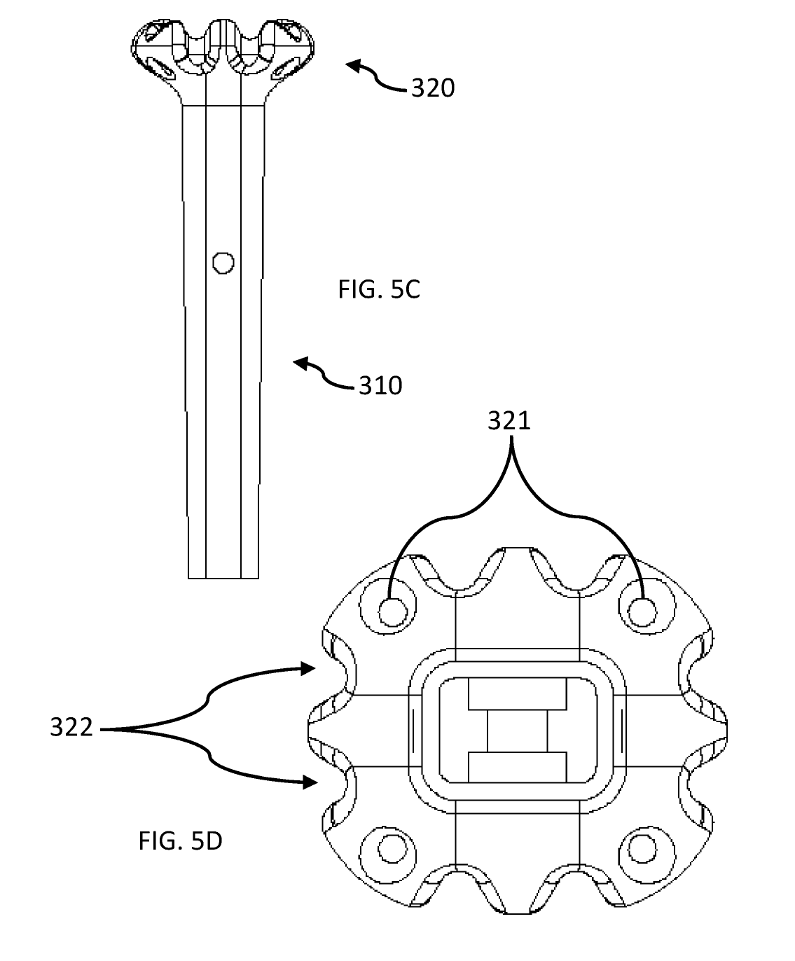

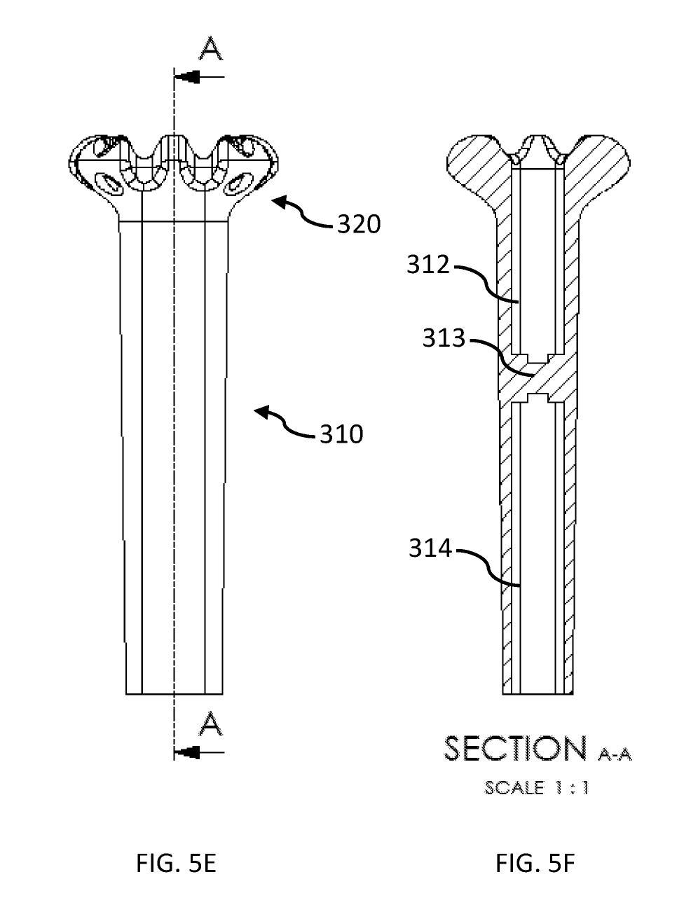

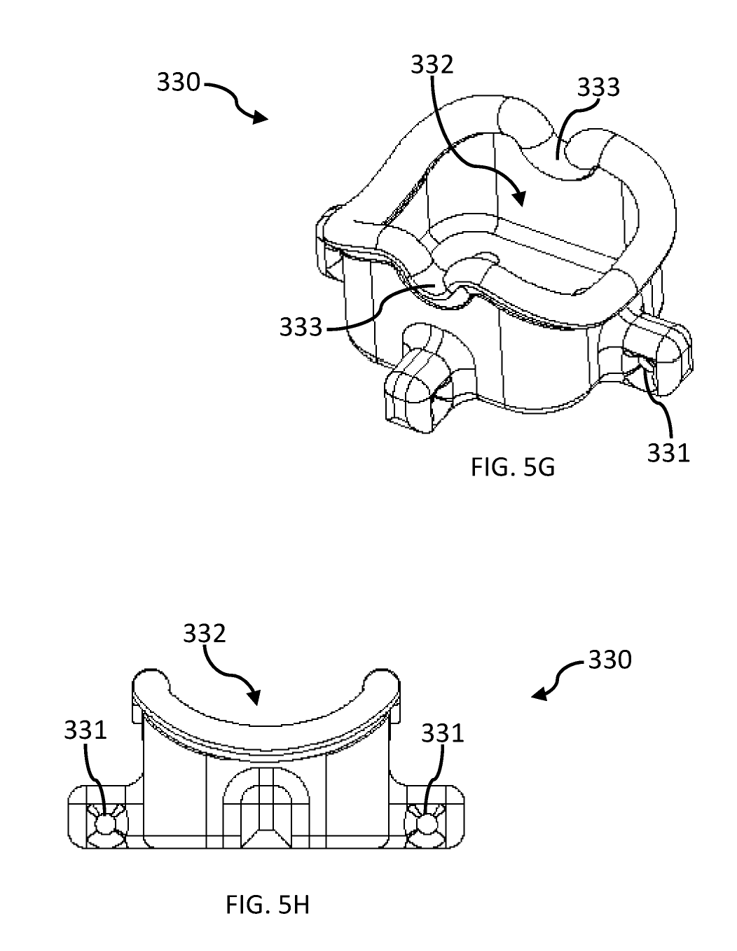

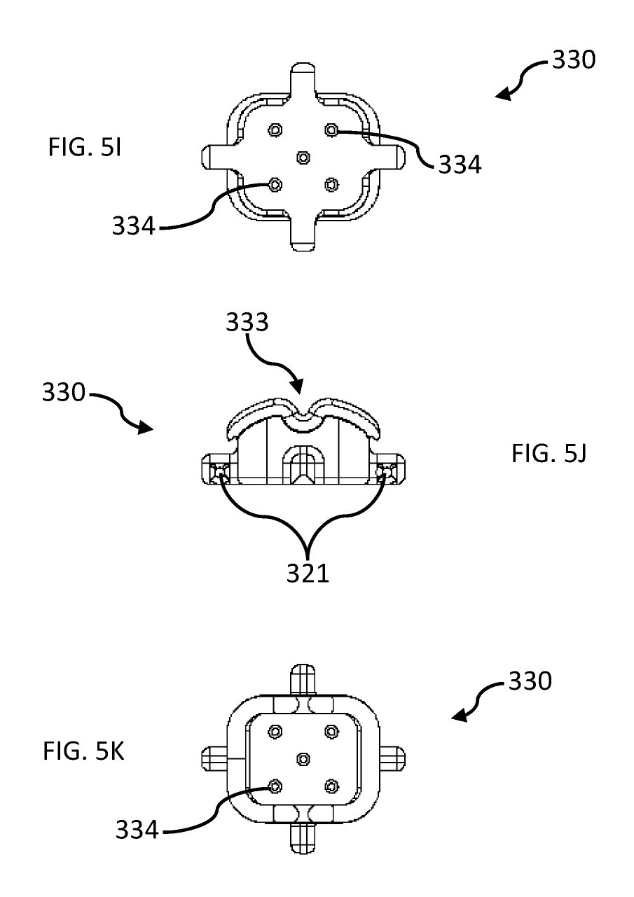

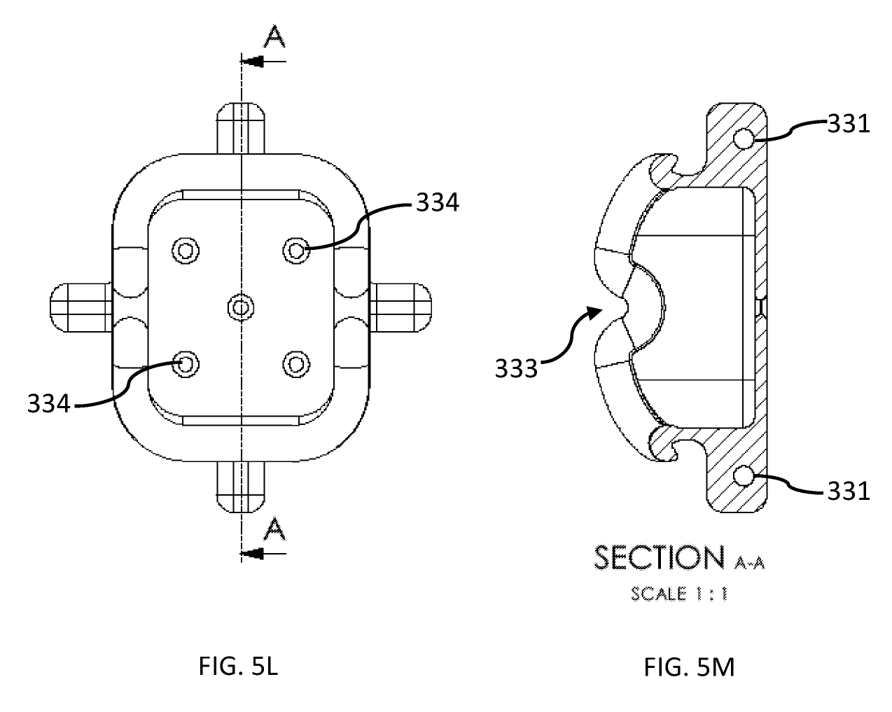

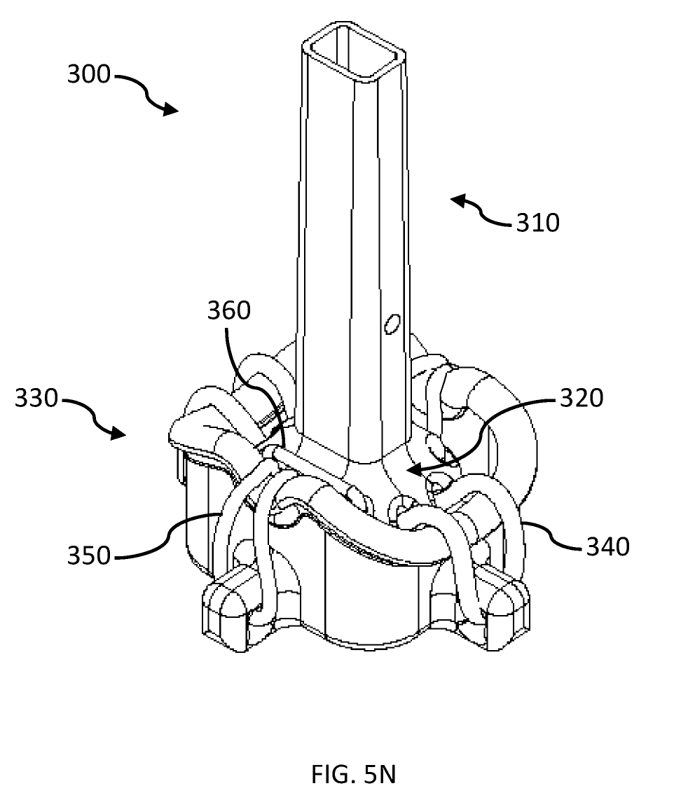

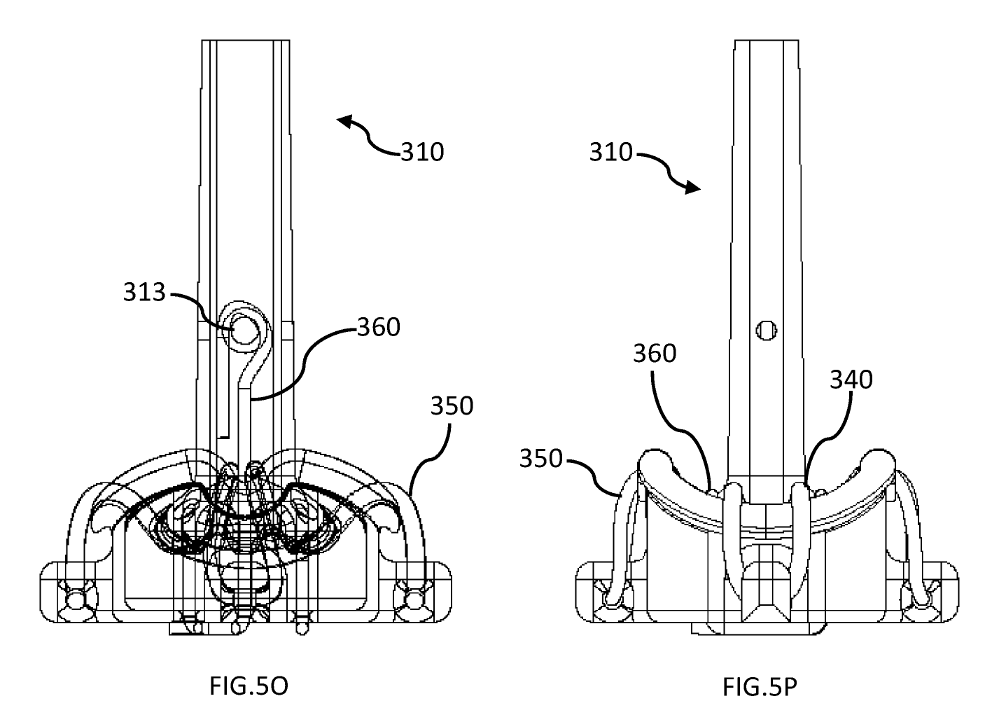

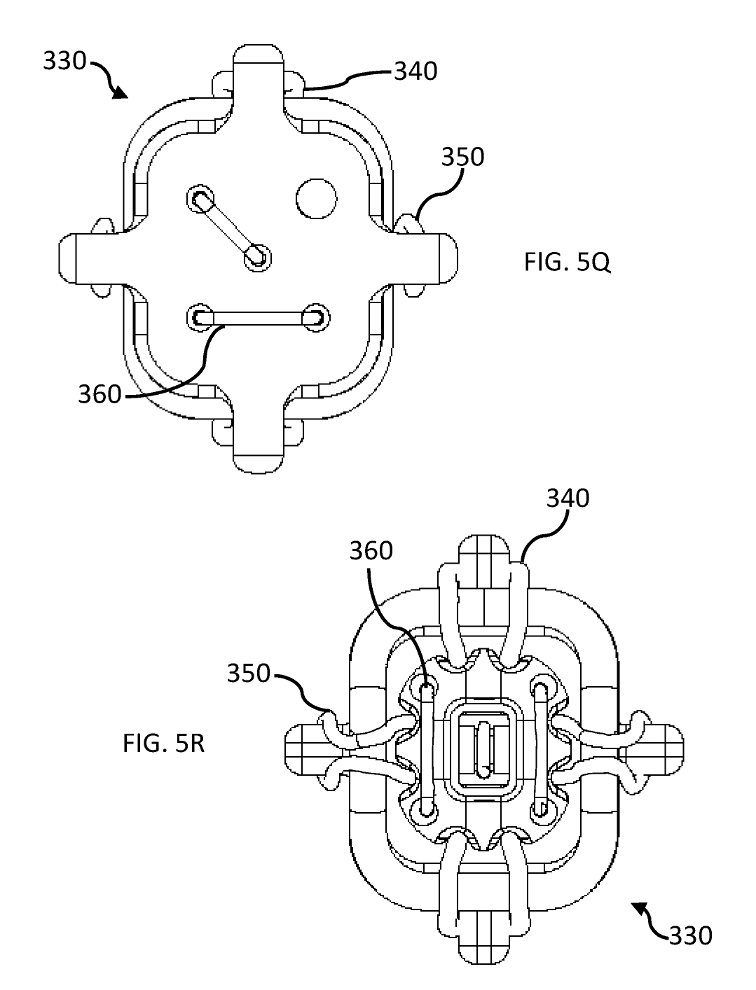

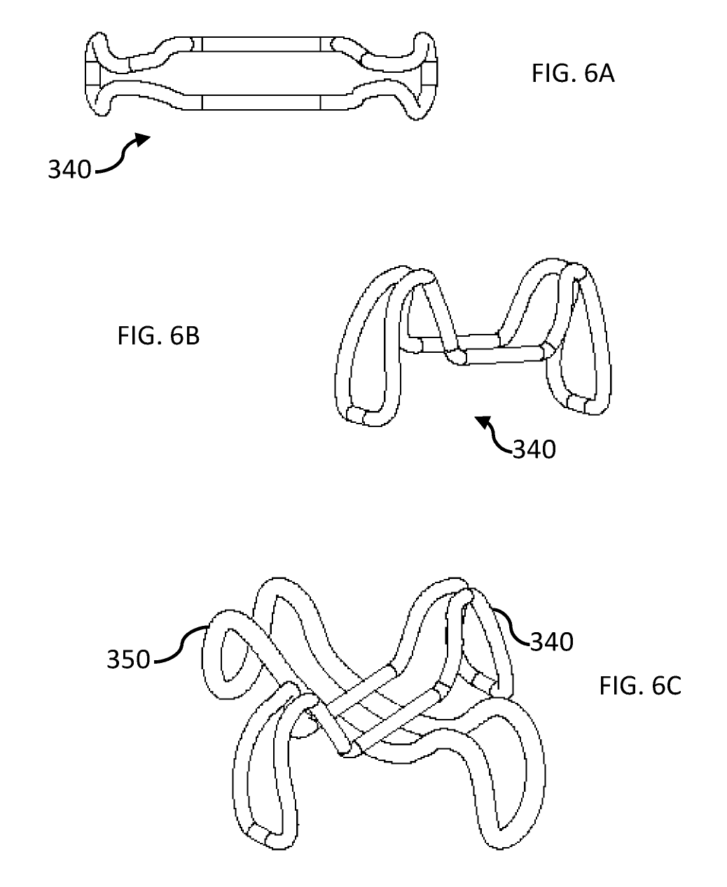

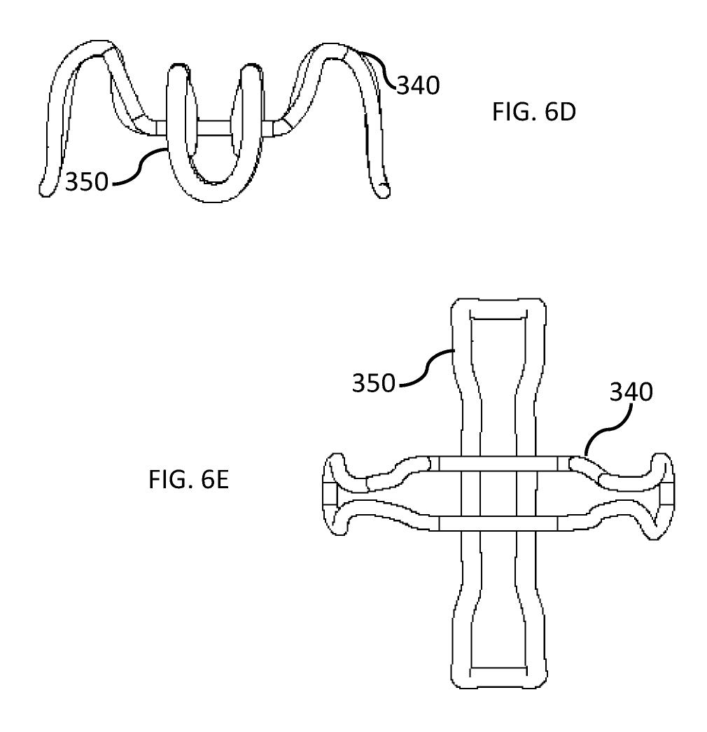

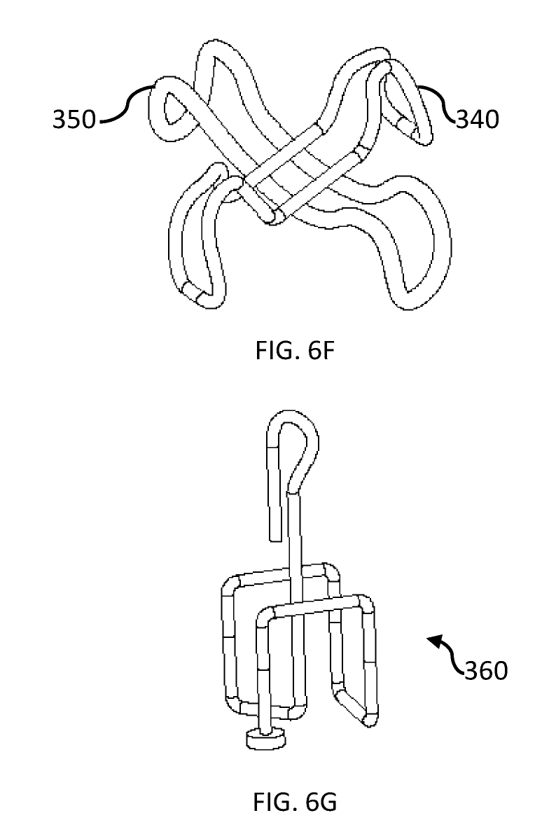

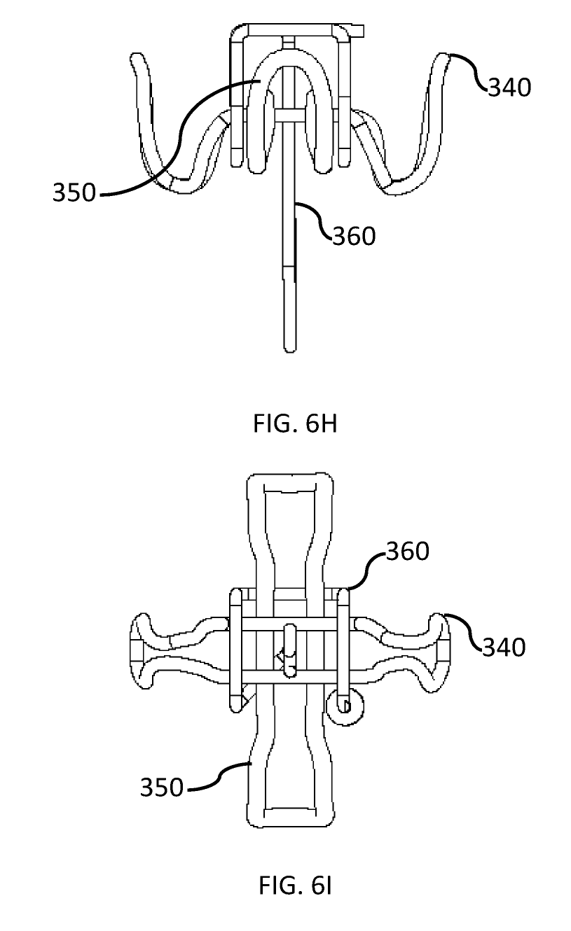

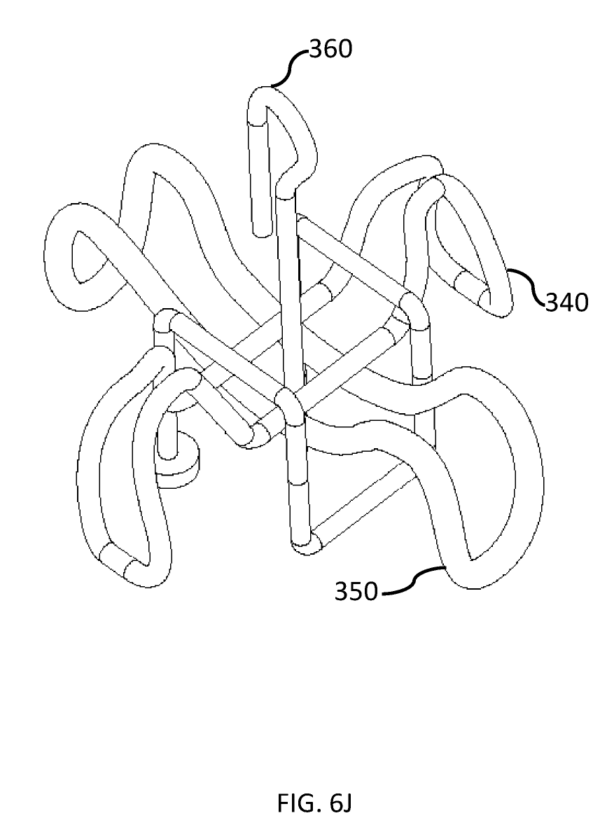

FIGS. 5-6 illustrate an example saddle joint 300 in accordance with embodiments of the present disclosure. Saddle joint 300 includes a socket component 330 and a shaft component having a shaft 310 and a head 320 configured for receipt within socket component 330. The upper edge of socket component 330 has a rounded lip feature so that one or more tension members wrapped over the edge are appropriately supported and are not abraided in use.

At least one tension member 360 couples head 320 to socket 330 and constrains their relative motion.

Since flexible tension members under load will always move to the lowest energy location, the lateral tension member has a locating feature built into the shape of the lip of the saddle joint socket. The saddle joint socket also includes attachment means for tension members, located on logical places on the intended path of the tension member.

While any of a variety of configurations may be implemented, in an example, tension member 360 may comprise several tension members. Referring to FIGS. 5N-5R, a first tension member 340 winds through a first peripheral anchor 331, through a first mediolateral channel 322, through a second peripheral anchor 331 opposite the first peripheral anchor and then through a second mediolateral channel 322 and forms a continuous loop. Channels 322 of head 320 glide on first tension member 340. In an example, first tension member 340 may be considered a saggital tension member. Channels 322 terminate in radii, meant to support wrap tension members wrapping around these radii without being damaged.

A second tension member 350 winds through a third peripheral anchor 331, through a first locating feature 333, through a first anteroposterior channel 323, through a second locating feature 333, through a fourth peripheral anchor 331 opposite the third peripheral anchor, back through the second locating feature 333, through a second anteroposterior channel 323, back through the first locating feature 333 and forms a continuous loop. In this arrangement, head 320 of the shaft component rides on the web formed by threading of the first and second tension members 340 and 350 through peripheral anchors 331 and across socket component 330. Channels 323 of head 320 glide on second tension member 350. In an example, second tension member 350 may be considered a frontal or lateral tension member.

The combined action of tension members 340 and 350 resist movement of head 320 and socket 330 from moving toward each other by forming a flexible barrier therebetween where each of the four lengths of the tension members are positively located by features on the two head and socket.

A third tension member 360 winds through an anteriomedial intermediate anchor 334 of socket 330, through an anteriomedial intermediate anchor 321 of head 320, through a posteriomedial intermediate anchor 321 of head 320, through a posteriomedial intermediate anchor 334 of socket 330, through a posteriolateral intermediate anchor 334 of socket 330, through a posteriolateral intermediate anchor 321 of head 320, through an anteriolateral intermediate anchor 321 of head 320, through an anteriolateral intermediate anchor 334 of socket 330, through a central anchor 334 of socket 330 and is secured to central anchor 313 of shaft 310. Third tension member 360 prevents distraction of head 320 from socket 330. In an example, third tension member 360 may be divided into multiple individual tension members configured for individual tuning such that motion of the shaft component relative to socket component 330 is constrained differently about different axes and/or directions. In another example, third tension member 360 may attach to the head 320, or both ends could attach to the saddle component 330.

Third tension member 360 restricts the movement of the head 320 away from socket component 330 while also creating a physical restraint system to inhibit the dislocation of the segments of the first and second members from locating features 322, 323 and 333t. As head 320 rocks and slides on the first and second tension members 340 and 350, tension member 360 slides along its own path, moving relative to all other parts of this joint. Third tension member 360 also serves to limit the range of motion of head 320 relative to socket comonent 330, as it is a fixed length and may be relatively inelastic.

In some examples, third tension member 360 may cross the joint in more or less places. Some examples may include a mechanism for changing the length or the tension in third tension member 360, allowing for control of the range of motion and/or the stiffness of the joint movement.

Movement of saddle joint 300 occurs in tipping, sliding and twisting modes and in combinations thereof. In a tipping motion, the head 320's lip rolls on one or both saddle tension members, without slipping or sliding on the tension member itself. In tipping, third tension member 360 moves relative to head 320 and socket component 330. In sliding, channels 322 and 323 of head 320 translate relative to first and second tension members 340 and 350, and third tension member 360 moves relative to head 320 and socket component 330. In twisting, head 320 is rotated around its longitudinal axis. Third tension member 360 does not move relative to head 320 or socket component 330. First and second tension members 340 and 350 do not slide in channels 322 or 323. The range of motion of twisting is a function of all tension members of joint 300. The tension members should be of a length such that they are reach the limits of their range of motion together and no single tension member bears a disproportionate load and early failures of the joint system are avoided.

Head 320 may take any of a roughly rectangular cross section, though other cross sectional shapes may function similarly. The socket component takes a shape conducive to receiveing head 320. With first and second tension members 340 and 350 orthogonal to one another, saddle joint 300 may be considered to be homokinetic joint. In another example, head 320 may be a parallelogram rather than a rectangle, and socket component 330 a corresponding shape such that the joints is heterokinetic. In an example, the first and second tension members can be 15 degrees over orthogonal which is similar to the angles of a human ankle/foot and would facilitate a gait similar to normal human locomotion.

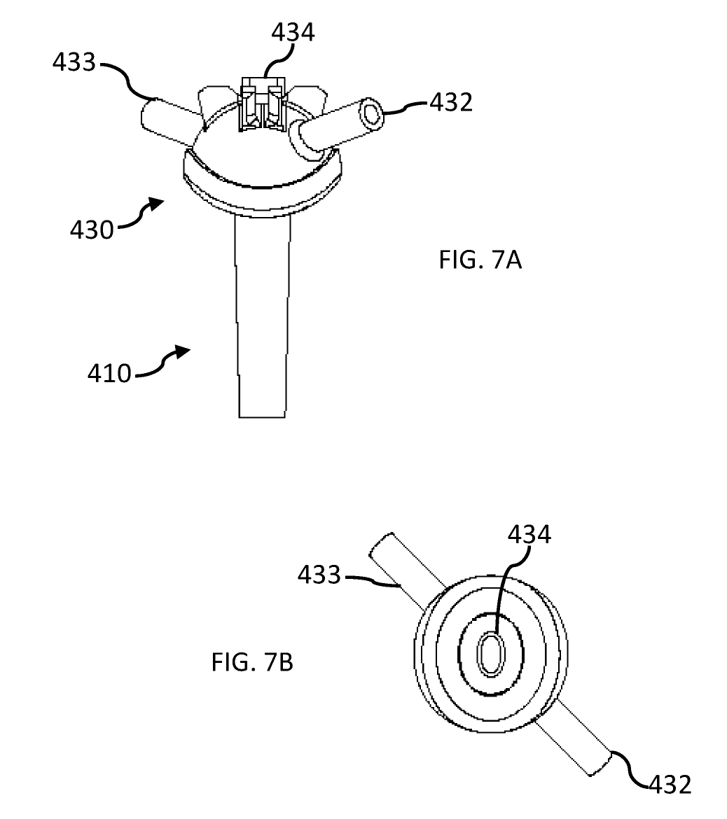

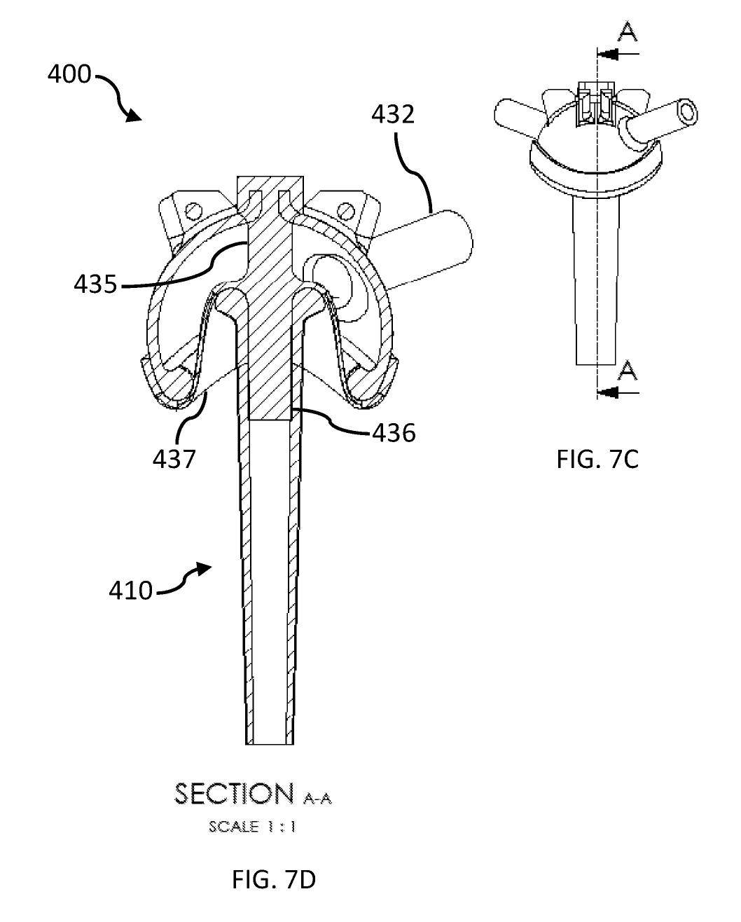

FIGS. 7A-7D illustrate an example tensegrity hip joint in accordance with embodiments of the present disclosure. Hip joint 400 includes a shaft component 410 configured for receipt within and articulation relative to socket component 430.

Shaft component 410 is coupled with socket component 430 by a fabric or fiber cloth wrapping tension member having an internal socket segment 435, an internal shaft segment 436 and an external socket segment 437. The tension member constrains the relative motion between shaft component 410 and socket component 430. The wrapping tension member may be bonded, laminated or clamped onto socket component 430 and shaft component 410. In an example, the wrapping tension member may be woven directly into the desired shape by CAM weaving, yielding a 3-dimensional shape that perfectly fits onto the geometry of the joint.

In an example, the wrapping tension member is establishes an airtight seal with socket component 430. Ports 432 and 433 enable introduction of fluid such as air to the chamber between socket component 430 and external socket segment 437 to apply internal pressure to external socket segment 437 and provide a tensile load to internal socket segment 435 and internal shaft segment 436. With dynamic control of joint stiffness or laxity by virtue of the air pressure within socket component 430 and the elastic component of the composite axial tension working against each other energy is stored and the joint is stiffened.

Axial components 435 and 436 of the wrapping tension member, which are attached with anchor 434, may be a composite construction with a flexible component that has a high elastic modulus, and another flexible component that is more elastic. The high modulus component would take a somewhat more circuitous route than the elastic component, thus the composite axial tension member would serve as a spring with a stop.

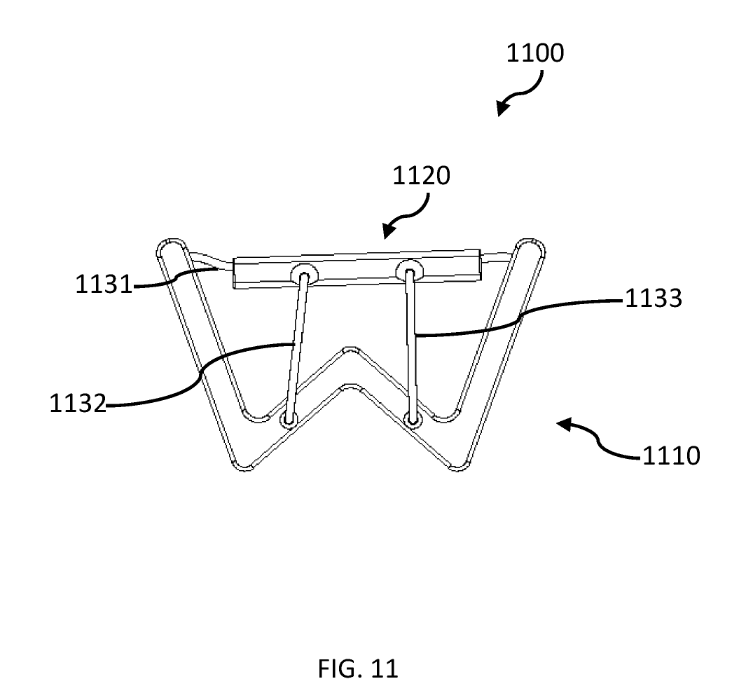

Referring to FIG. 11, a third example of a simplified geometry also includes a base member 1110, a suspended member 1120 and at least one tension member. In an example, the at least one tension member comprises a first tension member 1131, a second tension member 1132 and a third tension member 1133. Base member 1110 includes at least two base member peripheral anchors and also includes at least two base member intermediate anchors located between the base member peripheral anchors. Suspended member 1120 includes at least one lumen therethrough and also at least two suspended member intermediate anchors. First tension member 1131 couples the base member peripheral anchors to one another through the lumen of the suspended member, second tension member 1132 couples the first base member intermediate anchor to the first suspended member intermediate anchor and third tension member 1133 couples the second base member intermediate anchor to the second suspended member intermediate anchor so that base member 1110 and suspended member 1120 are configured for constrained relative articulation with suspended member 1110 partially received within base member 1120.

In another example, the third simplified geometry maybe multiplied and combined with itself. The at least two base member peripheral anchors include first, second, third and fourth base member peripheral anchors provided such that the first and third peripheral anchors are diametrically opposed and the second and fourth base member peripheral anchors are diametrically opposed. The at least two base member intermediate anchors include first, second, third and fourth base member intermediate anchors including diametrically opposed pairs analogous to those of the peripheral anchors. The at least one lumen includes first and second lumens. The at least two suspended member intermediate anchors include first, second, third and fourth suspended member intermediate anchors which may also include diametrically opposed pairs. The at least one tension member couples the first base member peripheral anchor with the third base member peripheral anchor through the first lumen, couples the second base member peripheral anchor with the fourth base member peripheral anchor through the second lumen, couples the first base member intermediate anchor with the first suspended member intermediate anchor, couples the second base member intermediate anchor with the second suspended member intermediate anchor, couples the third base member intermediate anchor with the third suspended member intermediate anchor, and couples the fourth base member intermediate anchor with the fourth suspended member intermediate anchor.

In another example combination, the third geometry may be combined with at least one other disclosed geometry. The at least two base member peripheral anchors include first, second, third and fourth base member peripheral anchors; the at least one base member intermediate anchor includes first, second and third base member intermediate anchors; the at least one suspended member intermediate anchor includes first, second and third suspended member intermediate anchors; and the suspended member further comprises a lumen therethrough. The at least one tension member couples the first base member peripheral anchor to a first of the suspended member peripheral anchors, couples the third base member peripheral anchor to a second of the suspended member peripheral anchors, couples the second base member peripheral anchor to the fourth base member peripheral anchor through the lumen of the suspended member, couples the first base member intermediate member with the first suspended member intermediate anchor, couples the first base member intermediate member with the first suspended member intermediate anchor and couples the first base member intermediate member with the first suspended member intermediate anchor.

Modifications to embodiments of the present disclosure described in the foregoing are possible without departing from the scope of the present disclosure as defined by the accompanying claims. Expressions such as “including”, “comprising”, “incorporating”, “consisting of”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A knee joint, comprising:
a patellar component including first, second, third and fourth patellar component peripheral anchors and first and second patellar component intermediate anchors located between the patellar component peripheral anchors;
a femoral component including at least one lumen therethrough and first and second femoral component intermediate anchors;
a tibial component including at least one lumen therethrough and first and second tibial component intermediate anchors;
at least one tension member coupling the first and second patellar component peripheral anchors to one another through the lumen of the femoral component, coupling the third and fourth patellar component peripheral anchors to one another through the lumen of the tibial component, coupling the first femoral component intermediate anchor to the first tibial component member intermediate anchor, coupling the second femoral component intermediate anchor with the second tibial component intermediate anchor so that the femoral component and the tibial component are configured for constrained articulation relative to the patellar component and are partially received thereby.

2. An articulating joint, comprising:
a base member including at least two base member peripheral anchors and a base member intermediate anchor located between the base member peripheral anchors;
a suspended member including at least two suspended member peripheral anchors and a suspended member intermediate anchor located between the suspended member peripheral anchors; and
at least one tension member coupling the base member peripheral anchors to the suspended member peripheral anchors and coupling the base member intermediate anchor to the suspended member intermediate anchor so that the base member and the suspended member are configured for constrained relative articulation with the suspended member partially received within the base member.

3. The articulating joint, as set forth in claim 2, wherein the at least one tension member includes a first tension member coupling a first of the at least two base member peripheral anchors to a first of the at least two suspended member peripheral anchors, a second tension member coupling a second of the at least two base member peripheral anchors to a second of the at least two suspended member peripheral anchors and a third tension member coupling the base member intermediate anchor with the suspended member intermediate anchor.

4. The articulating joint as set forth in claim 2, wherein the at least one tension member is configured to substantially prevent contact of the suspended member with the base member.

5. The articulating joint, as set forth in claim 2, wherein:
the at least two base member peripheral anchors include first, second, third and fourth base member peripheral anchors;
the at least two suspended member peripheral anchors include first, second, third and fourth suspended member peripheral anchors; and
the at least one tension member couples the first base member peripheral anchor to the first suspended member peripheral anchor, couples the second base member peripheral anchor to the second suspended member peripheral anchor, couples the third base member peripheral anchor to the third suspended member peripheral anchor, and couples the fourth base member peripheral anchor to the fourth suspended member peripheral anchor.

6. The articulating joint as set forth in claim 5, wherein the base member comprises an acetabular cup.

7. The articulating joint as set forth in claim 5, wherein the suspended member comprises a femoral head and shaft.

8. The articulating joint as set forth in claim 5, wherein the suspended member includes a plurality of protections configured to prevent sliding of the suspended member relative to the at least one tension member.

9. The articulating joint as set forth in claim 5, wherein the suspended member includes a plurality of notches configured to engage with the at least one tension member to constrain twisting of the suspended member relative to the base member about an axis directed from the center the suspended member to the center of the base member.

10. The articulating joint as set forth in claim 5, wherein twisting of the suspended member on the base member distracts the suspended member from the base member.

11. The articulating joint as set forth in claim 5, wherein the first and third base member peripheral anchors are substantially diametrically opposed and the second and fourth base member peripheral anchors are substantially diametrically opposed.

12. The articulating joint as set forth in claim 5, wherein the first and third suspended member peripheral anchors are substantially diametrically opposed and the second and fourth suspended member peripheral anchors are substantially diametrically opposed.

13. The articulating joint as set forth in claim 2, wherein:
the at least two base member peripheral anchors include first, second, third and fourth base member peripheral anchors;
the suspended member includes a lumen therethrough; and
the at least one tension member couples the first and third base member peripheral anchors with the at least two suspended member peripheral anchors and couples the second and fourth base member peripheral anchors to one another through the suspended member lumen.

14. The articulating joint as set forth in claim 2, wherein:
the at least two base member peripheral anchors include first, second, third and fourth base member peripheral anchors;
the at least one base member intermediate anchor includes first, second and third base member intermediate anchors;
the at least one suspended member intermediate anchor includes first, second and third suspended member intermediate anchors;
the suspended member further comprises a lumen therethrough; and
the at least one tension member couples the first base member peripheral anchor to a first of the suspended member peripheral anchors, couples the third base member peripheral anchor to a second of the suspended member peripheral anchors, couples the second base member peripheral anchor to the fourth base member peripheral anchor through the lumen of the suspended member, couples the first base member intermediate member with the first suspended member intermediate anchor, couples the first base member intermediate member with the first suspended member intermediate anchor and couples the first base member intermediate member with the first suspended member intermediate anchor.

15. An articulating joint, comprising:
a first suspended member including at least one lumen therethrough and a suspended member intermediate anchor;
a base member configured to receive a portion of the first suspended member and including at least two base member peripheral anchors and at least one base member intermediate anchor located between the base member peripheral anchors; and
at least one tension member threaded through the base member and suspended member intermediate anchors and coupling the base member peripheral anchors to one another through the lumen of the suspended articulating member so that the base member and the suspended member are configured for constrained relative articulation with the suspended member partially received within the base member.

16. The articulating joint as set forth in claim 15, further comprising a second suspended member including at least one lumen therethrough and a suspended member intermediate anchor and wherein the second suspended member is coupled, by the at least one tension member, with the base member opposite the first suspended member.

17. The articulating joint as set forth in claim 16, wherein the at least one tension member couples the intermediate anchor of the first suspended member with the intermediate anchor of the second suspended anchor.

18. The articulating joint as set forth in claim 16, wherein the first suspended member comprises a femoral component and the second suspended member comprises a tibial component.

19. The articulating joint as set forth in claim 15, wherein the first suspended member includes a second lumen and two intermediate anchors and the at least one tension member couples the base member peripheral anchors to one another through the second lumen.

20. The articulating joints as set forth in claim 15, wherein:
the at least two base member peripheral anchors include first, second, third and fourth base member peripheral anchors;
the at least one lumen includes first and second lumens; and
the at least one tension member couples the first base member peripheral anchor to the third base member peripheral anchor through the first lumen and couples the second base member peripheral anchor to the fourth base member peripheral anchor through the second lumen.

21. The articulating joint as set forth in claim 15, wherein:
the at least two base member peripheral anchors include first, second, third and fourth peripheral anchors;
the at least one base member intermediate anchor includes first, second and third base member intermediate anchors;
the at least one lumen includes first and second lumens;
the at least one suspended member intermediate anchor includes first, second and third suspended member intermediate anchors; and
the at least one tension member couples first and third base member peripheral anchors to one another through the first lumen, couples the second and fourth base member peripheral anchors to one another through the second lumen, couples the first base member intermediate anchor with the first suspended member intermediate anchor, couples the second base member intermediate anchor with the second suspended member intermediate anchor and couples the third base member intermediate anchor with the third suspended member intermediate anchor.
